(19) United States
(12) Patent Application Publication
(10) Pub. No.: US 2022/0148689 A1
(43) Pub. Date: May 12, 2022

(54) AUTOMATICALLY PRE-CONSTRUCTING A CLINICAL CONSULTATION NOTE DURING A PATIENT INTAKE/ADMISSION PROCESS

(71) Applicant: OneLine Health LLC, Pasadena, CA (US)

(72) Inventors: Karanvir S. Gill, Pasadena, CA (US); Jamal A. Nabhani, Pasadena, CA (US); Inderbir S. Gill, Pasadena, CA (US)

(21) Appl. No.: 17/500,729

(22) Filed: Oct. 13, 2021

Related U.S. Application Data
(60) Provisional application No. 63/091,583, filed on Oct. 14, 2020.

(51) Int. Cl. G16H 10/20; G16H 10/60; G16H 30/20

(52) U.S. Cl. CPC G16H 10/20; G16H 30/20; G16H 10/60

(57) ABSTRACT

Systems, methods, and apparatus for automatically pre-constructing a clinician consultation note during a patient intake or admission process are disclosed herein. The example systems, methods, and apparatus are configured to automatically populate a clinical consultation note by extracting relevant medical information from unstructured past medical documentation in addition to incorporating patient data from an active sheet comprising a medical questionnaire form. The active sheet is configured to acquire structured patient data from a patient through the use of defined data entry fields. The active sheet is programmed with logic such that selection of some data entry fields will cause the inclusion of additional data entry fields. The logic is defined to replicate a physician's own medical training to prompt patients for more detailed patient data regarding a specific medical condition in an automated manner. Such a configuration is more efficient and ensures that key medical questions are not overlooked.

### PRIORITY CLAIM

This application claims priority to and the benefit as a non-provisional application of U.S. Provisional Patent Application No. 63/091,583, filed Oct. 14, 2020, the entire contents of which are hereby incorporated by reference and relied upon.

### BACKGROUND

When a patient meets with a physician, the patient has to proceed through a structured intake or admission procedure of a medical system. While each medical system has their own specific procedures, the overall process is generally the same. In particular, known intake or admission procedures require patients to complete a medical questionnaire form online or in a medical office. The form is generally static and prompts patients for very basic and high-level information, such as their demographic information (e.g., race, gender, ethnicity, etc.), generic medical history, current/past medical prescriptions, and a reason a patient is requesting a visit with a physician. The form sometimes includes tens of symptoms for possible selection.

In addition to the medical questionnaire form, the patient intake procedure also includes acquiring past medical documentation. This includes laboratory results, x-rays or other medical device scans, prior clinical notes, and/or a patient's medical record, which could include an electronic medical record (“EMR”). Oftentimes, the past medical documentation comes from the patient themselves. However, more likely, the past medical documentation is transmitted from other medical systems from which the patient has received medical treatment. Given the extreme fragmentation and regulatory constraints around patient privacy in the medical industry, the past medical documentation is not usually sent as electronic source files, but rather scans of physical documents or read-only electronic files.

As one can appreciate, known patient intake procedures only compile the past medical tests, documentation and information from the medical questionnaire form for a physician to review. For patients with extensive medical histories, this can easily translate into tens to hundreds of pages of medical documentation. A physician can easily consume approximately thirty minutes, if not an hour or more, reviewing all this information. Further, given all this fragmented medical documentation, it is possible for a physician to overlook at least some of the patient's medical history.

In addition to reviewing the medical documentation, as part of the patient intake, the physician must summarize all the salient points from the medical documentation in a clinical consultation note. Constructing a clinician consultation note can take anywhere from fifteen minutes to over an hour depending on the extent of the past medical documentation and complexity of the patient's condition. Time spent preparing the note often takes away time the physician could have spent personally interacting with the patient or other patients. During a patient visit, the physician completes the clinical consultation note. Typically, during an actual patient visit, a physician spends as much time reviewing prior medical documentation and transcribing essential details therefrom (both repetitive tasks), as the time they spend interacting with the patient and making treatment plans. Further, the time spent transcribing the clinical consultation note detracts from the patient experience, and also this repetitive, scribe-like activity with the electronic medical records (EMR) system contributes to physician burn-out.

### SUMMARY

Systems, methods, and apparatus for automatically pre-constructing a clinician consultation note during a patient intake or admission process are disclosed herein. The example systems, methods, and apparatus are configured to automatically populate a clinical consultation note by extracting relevant medical information from past medical documentation, in addition to incorporating patient data from an “active sheet” comprising a medical questionnaire form with built-in logic. The active sheet is configured to acquire structured patient data from a patient through the use of defined data entry fields. The active sheet is programmed with logic such that selection of some data entry fields subsequently causes the sequential inclusion of additional data entry fields. The logic is defined to replicate a physician's own medical training, clinical reasoning, and decision-making to prompt patients to provide more detailed patient data regarding a specific medical condition in an automated manner. Such a configuration is more efficient and ensures that key medical questions are not overlooked.

In an example, the active sheet disclosed herein prompts a patient for general symptoms. After selection of symptoms, logic in the active sheet is configured to determine further questions to add to the active sheet in the form of specific data fields. Selection, for example, of a “blood in urine” symptom causes logic in the active sheet to display only pertinent questions and related data fields prompting a patient for more specific, in-depth questions related to the urologic history for “blood in urine”. The active sheet is configured to cover virtually any medical condition to provide detailed comprehensive clinical history of the patient for automatic population into the clinical consultation note. For patients, active sheets contain no medical jargon and are displayed in common everyday terms that non-medical professionals are able to understand. Conversely, when populating the clinical consultation note for medical providers, these lay, everyday terms are converted and presented in the typical medical terminology that is commonly familiar to all medical professionals.

In some embodiments, a format or template for the clinical consultation note is selected based on an identified medical condition. In these embodiments, the clinician consultation note has sections that are relevant to the specific medical condition of the patient rather than general sections that cover all medical conditions. For example, medical conditions related to broken bones may have sections for x-rays while internal organ issues may have sections for magnetic resonance imaging (“MRI”) or computed tomography (“CT”) images, and medical conditions related to diseases may include “laboratory test” result sections.

The example systems, methods, and apparatus are also configured to automatically construct the clinical consultation note by extracting relevant medical information from past medical documentation. As mentioned above, some of the medical documentation may comprise scanned pages. In these instances, the systems, methods, and apparatus are configured to use one or more text extraction routines for identifying and/or modifying relevant medical information for inclusion within the clinical consultation note.

In addition to constructing a clinical consultation note, the example systems, methods, and apparatus are configured to link or otherwise associate information in the clinical consultation note with a source of the information. For example, a section of a clinical consultation note may automatically populate laboratory results from prior documentation in a physician-friendly format. Selection of this section by a physician causes the example systems, methods, and apparatus to retrieve a linked EMR document that shows the basis of the laboratory results. Such a configuration provides transparency and enables a physician to easily and immediately view sources of medical information without having to manually locate the document in a stack of scanned medical documents. The links may be provided for the section as a whole or may be provided for certain words or other medical information.

The example clinical consultation note may include color coding that provides a workflow for a physician. The color coding may specify areas that require a physician's attention either before a patient visit or during a patient visit where more patient data may be needed. Information on the clinical consultation note may be colored or highlighted in yellow or red to indicate that the clinician should review. Selection of this information (or section) in the clinical consultation note causes the systems, methods, and apparatus to remove the color coding or change the color to a different color, such as grey or green. Blue areas may correspond to sections that need more information and are configured to change color after the information is entered.

The clinical consultation note further includes sections that are completed by a physician after a meeting with a patient. In some embodiments, the clinical consultation note is configured to enable a physician to drag or otherwise copy patient data from other sections for inclusion in a clinical summary/meeting notes/recommended actions section. In some instances, the systems, methods, and apparatus disclosed herein use natural language processing of a free-text entry within an active sheet or a voice recording of the physician-patient interaction (either in person or via teleconferencing) to automatically update the clinical summary section.

The example systems, methods, and apparatus may also provide for automated billing for the patient intake/admissions process. Specifically, the systems, methods, and apparatus are configured to record how much time a physician spent reviewing/editing a clinical consultation note using a timer system. The time may also include an amount of pre-visit time spent trying to connect with the patient by phone or tele-video, in addition to the time spent during the actual consultation meeting with the patient. Additionally, the clinical consultation note may include a section that enables a clinician to select relevant Medical Decision Making complexity categories and criteria. The systems, methods, and apparatus may determine whether a time-based requirement was satisfied, and if so, a first billable amount. The systems, methods, and apparatus also generate a second billing amount related to the selected medical decision making categories and criteria. The systems, methods, and apparatus are configured to select the greater billable amount to provide for automatic patient/insurance billing that is in compliance with medical insurance rules/guidelines, thereby optimizing physician billing capabilities in a definitive manner.

In some embodiments, the timer system is configured to provide an aggregate time spent on the patient's visit by adding three specific time segments: a) a pre-visit time spent in filling out the active sheet by the medical provider's team; b) an intra-visit time spent during the actual consultation encounter including finalizing the clinical consultation note; and c) apost-visit time spent preparing the patient's clinical note in the EMR. The timer system is connected to a color-coded paragraph on Medical Decision Making complexity, which will change color from, for example, yellow or red (which indicates that the clinician should review this paragraph), to for example, grey or green or remove the coloring after the physician has reviewed it. After review, the system enables billing based on the timer system. Such a configuration ensures accuracy in billing, maximizing billing, and prevents billing if the physician has not personally reviewed that paragraph.

The systems, methods, and apparatus accordingly provide an automated patient intake/admission procedure that combines structured subjective patient data from an active sheet questionnaire form and unstructured objective patient data from past medical laboratory and radiological imaging test documentation to automatically construct a clinical consultation note. The automation of the systems, methods, and apparatus ensures that patient data and medical information are accurately recorded without errors in an efficient manner, with little or no manual input from a physician or other health care provider. The systems, methods, and apparatus enable medical documentation to be conducted in the background, prior to the clinic visit, which enables physicians to spend more time engaging with patients, thereby improving patient satisfaction, physician satisfaction, and the overall health care patient experience.

In light of the disclosure set forth herein, and without limiting the disclosure in any way, in a first aspect of the present disclosure, which may be combined with any other aspect, or portion thereof, described herein a system for automatically pre-constructing a clinical consultation note during a patient intake/admission process includes a memory device storing an active sheet including a medical questionnaire form having data fields that prompt a patient to select symptoms from among a plurality of symptoms, The active sheet includes embedded logic configured to select detailed medical questions based on the selection of symptoms, the detailed medical questions corresponding to one or more possible disease conditions, and determine tests or medical documentation needed for a consultation with a physician. The example system also includes a server communicatively coupled to the memory device. The server is configured to receive a request from a patient device to schedule a medical appointment with a physician, transmit the active sheet to the patient device after receiving the request, receive patient data from the patient device via the active sheet, the patient data including a selection of symptoms and answers to the detailed medical questions, and receive medical documentation via the active sheet. The server is also configured to extract relevant medical information from the medical documentation, the medical information including at least one of test results, medical images, clinician notes, prior treatments, or medical history information, generate automatically a clinical consultation note using the extracted relevant medical information and at least some of the patient data from the active sheet, and transmit the clinical consultation note to a physician device for the medical appointment with the patient.

In a second aspect of the present disclosure, which may be combined with any other aspect listed herein, the server is further configured to receive, via the active sheet, information indicative of another medical provider, transmit a request for third-party medical documentation to the other medical provider, receive the third-party medical documentation from the other medical provider, and extract relevant medical information from the third-party medical documentation for inclusion within the clinical consultation note.

In a third aspect of the present disclosure, which may be combined with any other aspect listed herein, the active sheet is configured to prompt a patient for additional patient data including at least one of past medical history, past surgical history, current/past medications, current/past allergies, family and social history, history of present illness, or general activity level.

In a fourth aspect of the present disclosure, which may be combined with any other aspect listed herein, the server is further configured to select the detailed medical questions embedded within the active sheet also using the additional medical information.

In a fourth aspect of the present disclosure, which may be combined with any other aspect listed herein, the test or medical documentation includes at least one of x-rays, patient fluid laboratory results, blood test laboratory results, computed tomography (“CT”) images and/or reports, magnetic resonance imaging (“MRI”) images and/or reports, ultrasound images and/or reports, previous clinical notes, or an electronic medical record (“EMR”).

In a sixth aspect of the present disclosure, which may be combined with any other aspect listed herein, the patient device includes an application configured as the active sheet, the application configured to receive the patient data from the patient and transmit the patient data to the server via one or more application programming interfaces.

In a seventh aspect of the present disclosure, which may be combined with any other aspect listed herein, the clinical consultation note includes sections for a review of patient symptoms, identification of a referring physician, identification of a pharmacy, laboratory results, medical images, prior clinical notes, a diagnosis/assessment to be completed by the physician, a principal plan to the completed by the physician, additional tests to be completed or selected by the physician, a discussion of risks benefits to be completed by the physician, an impression of medical condition, or action items to be completed.

In an eighth aspect of the present disclosure, which may be combined with any other aspect listed herein, the server is configured to receive the clinical consultation note from the physician device with sections completed by the physician and at least one action item selection, and store the clinical consultation note to a medical record associated with the patient.

In a ninth aspect of the present disclosure, which may be combined with any other aspect listed herein, the server is further configured to cause a medical system to perform the at least one action item selected.

In a tenth aspect of the present disclosure, which may be combined with any other aspect listed herein, the at least one action item includes at least one of a medication order, a request to send the clinical consultation note to another physician, an order for a laboratory test, a schedule for a next appointment, a request to send standardized written materials related to a medical condition to the patient device, or a request to submit a bill.

In an eleventh aspect of the present disclosure, which may be combined with any other aspect listed herein, the server is further configured to cause at least some of the sections of the clinical consultation note to be colored coded, and remove or change the color coding after detecting or receiving an indication that the section was selected or reviewed by the physician.

In a twelfth aspect of the present disclosure, which may be combined with any other aspect listed herein, the server is further configured to generate automatically the clinical consultation note by embedding at least some links in the clinical consultation note to the related extracted relevant medical information, wherein selection of an embedded link causes the related extracted relevant medical information to be displayed in conjunction with the clinical consultation note.

In a thirteenth aspect of the present disclosure, which may be combined with any other aspect listed herein, the server is further configured to generate automatically the clinical consultation note by embedding at least some links in the clinical consultation note to the at least some of the patient data from the active sheet.

In a fourteenth aspect of the present disclosure, which may be combined with any other aspect listed herein, the server is further configured to extract the relevant medical information from the medical documentation by performing one or more of the following functions: denoise the medical documentation, find number patches in the medical documentation, cut patches from the medical documentation, identify a denoised image in the medical documentation, remove isolated parts of the medical documentation, crop redundant or unneeded parts of the medical documentation, add a boundary to the medical documentation, or extract features from the medical documentation.

In a fifteenth aspect of the present disclosure, which may be combined with any other aspect listed herein, the server is further configured to extract the relevant medical information from the medical documentation by performing one or more of the following functions: remove black boxes, remove a table border, remove dots, or de-skew at least portions of the medical documentation.

In a sixteenth aspect of the present disclosure, which may be combined with any other aspect listed herein, the server is further configured to extract the relevant medical information from the medical documentation by modifying words and punctuation by deleting non-sense words, correcting spelling, or correcting punctuation.

In a seventeenth aspect of the present disclosure, which may be combined with any other aspect listed herein, at least some of the patient data is provided by a peripheral device that is communicatively coupled to the patient device, the peripheral device including at least one of a fitness tracker, a smartwatch, a smart-ring, a wireless weight scale, or a blood pressure cuff.

In an eighteenth aspect of the present disclosure, which may be combined with any other aspect listed herein, the server is further configured to use a timer system to determine an aggregate time spent a) pre-visit filling out the active sheet on behalf of the patient, b) intra-visit during the actual medical appointment with the patient, and c) post-visit spent completing the clinical consultation note, and cause a bill to be generated based on the aggregate time.

In a nineteenth aspect of the present disclosure, which may be combined with any other aspect listed herein, the server is further configured to receive a second request from the patient device to schedule a subsequent medical appointment with the physician, and use at least some of the patient data to pre-construct the active sheet.

In a twentieth aspect of the present disclosure, which may be combined with any other aspect listed herein, the server is further configured to receive additional medical documentation from a remote electronic medical record system via interoperability.

In a twenty-first aspect of the present disclosure, which may be combined with any other aspect listed herein, a method for automatically pre-constructing a clinical consultation note during a patient intake/admission process includes receiving, in a server, a request from a patient device to schedule a medical appointment with a physician, and transmitting, from the server to the patient device, an active sheet after receiving the request. The active sheet is stored in a memory device and includes an electronic medical questionnaire form having data fields that prompt a patient to select symptoms from among a plurality of symptoms. The active sheet includes embedded logic configured to select detailed medical questions based on the selection of symptoms, the detailed medical questions corresponding to one or more possible disease conditions, and determine tests or medical documentation needed for a consultation with a physician. The example method also includes receiving, in the server, patient data from the patient device via the active sheet, the patient data including a selection of symptoms and answers to the detailed medical questions, receiving, in the server, medical documentation via the active sheet, and extracting, via the server, relevant medical information from the medical documentation, the medical information including at least one of test results, medical images, clinician notes, prior treatments, or medical history information. The example method further includes generating automatically, via the server, a clinical consultation note using the extracted relevant medical information and at least some of the patient data from the active sheet, and transmitting, via the server, the clinical consultation note to a physician device for the medical appointment with the patient.

In a twenty-second aspect of the present disclosure, which may be combined with any other aspect listed herein, the method further includes receiving, in the server via the active sheet, information indicative of another medical provider, transmitting, from the server, a request for third-party medical documentation to the other medical provider, receiving, in the server, the third-party medical documentation from the other medical provider, and extracting, via the server, relevant medical information from the third-party medical documentation for inclusion within the clinical consultation note.

In a twenty-third aspect of the present disclosure, which may be combined with any other aspect listed herein, the method further includes receiving, in the server, the clinical consultation note from the physician device with sections completed by the physician and at least one action item selection, storing, via the server, the clinical consultation note to a medical record associated with the patient, and causing, via the server, a medical system to perform the at least one action item selected, wherein the at least one action item includes at least one of a medication order, a request to send the clinical consultation note to another physician, an order for a laboratory test, a schedule for a next appointment, a request to send standardized written materials related to a medical condition to the patient device, or a request to submit a bill.

In a twenty-fourth aspect, any of the features, functionality and alternatives described in connection with any one or more of FIGS. 1 to 37 may be combined with any of the features, functionality and alternatives described in connection with any other of FIGS. 1 to 37.

In light of the present disclosure and the above aspects, it is therefore an advantage of the present disclosure to provide an active sheet questionnaire with embedded logic that displays more detailed medical questions to a patient based on one or more selected symptoms.

In light of the present disclosure and the above aspects, it is therefore an advantage of the present disclosure to automatically generate a clinician consultation note as part of a patient intake/admission process using structured patient data from a questionnaire and unstructured medical information from medical documentation.

It is another advantage of the present disclosure to use patient data from an active sheet questionnaire to provide real-time diagnostic planning and investigations, and treatment planning for a patient.

It is another advantage of the present disclosure to automatically perform action items such as ordering diagnostic tests, sending a follow-up note to a referring physician(s), sending prescriptions to a pharmacy, providing guidelines documents that discuss disease progression/prognosis and/or guidelines for diet, activity, etc., or scheduling a follow up treatment after submission of a complete clinician consultation note.

It is yet another advantage of the present disclosure to automatically attach the correct CPT codes and bill the healthcare insurance group for medical billing reimbursement based on a physician's interaction and completion of the clinician consultation note.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein, and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 illustrates a diagram of a clinical consultation note system for creating a clinical consultation note using a management server, according to an example embodiment of the present disclosure.

FIGS. 2 to 4 are diagrams that are illustrative of a process flow for creating a clinical consultation note, according to an example embodiment of the present disclosure.

FIGS. 5 to 7 are diagrams of a clinical consultation note after generation by the management server of FIG. 1, according to an example embodiment of the present disclosure.

FIG. 8 is a diagram of a patient registration user interface for obtaining an active sheet questionnaire, according to an example embodiment of the present disclosure.

FIG. 9 is a diagram of a patient dashboard user interface with an option to select an active sheet questionnaire, according to an example embodiment of the present disclosure.

FIGS. 10 to 21 are diagrams that show different pages or user interfaces of an active sheet questionnaire for creating a clinical consultation note, according to an example embodiment of the present disclosure.

FIG. 22 is a diagram of the patient dashboard user interface of FIG. 9 after the active sheet questionnaire has been completed, according to an example embodiment of the present disclosure.

FIG. 23 is a diagram of a table that correlates recommended medical tests to symptoms for logic or algorithms of the active sheet questionnaire, according to an example embodiment of the present disclosure.

FIG. 24 shows a rule for selecting a medical test, according to an example embodiment of the present disclosure.

FIGS. 25 and 26 are diagrams that illustrate the logic of the active sheet questionnaire, according to example embodiments of the present disclosure.

FIG. 27 is a flow diagram of an example procedure to create a clinical consultation note using structured patient data and unstructured medical documentation, according to an example embodiment of the present disclosure.

FIGS. 28 to 36 are diagrams of example processes for extracting relevant medical information from unstructured medical documentation, according to an example embodiment of the present disclosure.

FIG. 37 is a diagram of a chart that shows a time requirement for certain billing labels and corresponding statements used by the management server of FIG. 1, according to an example embodiment of the present disclosure.

### DETAILED DESCRIPTION

Systems, methods, and apparatus for automatically pre-constructing a clinician consultation note during a patient intake or admission process are disclosed herein. The example systems, methods, and apparatus are configured to automatically generate a clinical consultation note using patient data acquired through an active sheet questionnaire and/or medical information extraction of prior medical documentation. The auto-completion of the clinical consultation note provides a comprehensive summary of a patient's medical condition and medical history in one unique document that would otherwise take a physician or other health care work up to one or two hours to prepare as part of a patient intake/admission procedure.

Current known patient intake procedures are manual and time-consuming. In a typical example, a physician's office asks a patient to provide their prior medical records and other documentation, usually one week before a physician consultation. This requires that a patient fill out a static questionnaire and provide copies of medical records. In some instances, the patient has to provide names and addresses of other physicians as well as sign medical record release waivers granting transfer of one's medical record so that the physician's office can obtain the medication documentation themselves. After the medical information and medical documentation is collected, a physician or medical staff review the information and determine which of the information is to be entered into a clinical consultation note. Time is also spent manually entering the identified information into the clinical consultation note. While some notes may comprise templates with defined sections, it still consumes considerable time locating, arranging, and entering all of the relevant information.

After a clinical consultation note is prepared, a physician meets with a patient to discuss a chief complaint and history of a present illness. Unfortunately, this time is limited to less than ten or fifteen minutes. During this time, the physician also discusses medications, allergies, past medical history, past surgical history, social history, family history, and/or occupation. After the meeting, the physician manually updates the clinical consultation note based on the meeting and examination of the patient. During this time, the physician also makes a final diagnosis, formulates a management plan, and orders any additional tests or procedures. For most consultations, this time can take ten to fifteen minutes. Additionally, the physician may obtain and give the patient guidelines that discuss a progression/prognosis of their disease and/or guidelines for diet, activity, etc. In some instances, the physician also schedules a next appointment, thereby inconveniencing the patient with an unsatisfactory initial consultation.

The example systems, methods, and apparatus overcome the manual and time-consuming patient intake/admission process discussed above by automatically generating and completing the clinical consultation note. The disclosed systems, methods, and apparatus enables clinicians to focus on higher-level real-time activities, such as, but not limited to, clinical assessment and diagnosis, clinical management decision-making, formulating treatment plans, patient education, and counseling. The systems, methods, and apparatus accordingly minimize and/or eliminate the need for clinicians to do mundane ‘scribe-like’ repetitive tasks, such as data abstracting and mining from source medical documentation to construct a concise yet comprehensive clinical note. For physicians, the example systems, methods, and apparatus provide for greater clinical productivity with higher patient throughput, freeing them for other clinical/administrative duties. In addition, since physicians will be able to spend more time in face-to-face interaction with patients, the systems, methods, and apparatus disclosed herein should improve patient satisfaction and engagement and facilitate a more meaningful medical consultation from the perspective of both the patient and the provider.

The example systems, methods, and apparatus are also configured to reduce potential systematic waste and medical documentation errors. As discussed above, medical documentation is oftentimes an unstructured data source of scanned documents or text files. The systems, methods, and apparatus are configured to scan the medical documentation for relevant medical information for inclusion into the clinical consultation note. In some embodiments, the systems, methods, and apparatus may improve readability of the medical information by correcting typographic errors, correcting grammar/spelling, deciphering handwriting, and improving overall visual quality of scanned documents.

Reference is made therein to medical information. As discussed in more detail below, medical information refers to information that is contained within medical documents of laboratory, radiologic and other such tests and investigations. This medical information is objective and generally provided in an unstructured format, or at least unstructured in relation to a clinical consultation note. The medical information may include x-rays, pictures, fluid laboratory results, blood test laboratory results, computed tomography (“CT”) images, magnetic resonance imaging (“MRI”), ultrasound images, previous clinical notes, or an electronic medical record (“EMR”). The medical documentation may include any document or file that contains medical information, such as a patient medical record or patient EMR, discharge notes, medical evaluation notes, etc. The medical documentation may include scans or photos of original documents, copies of original documents, or electronic versions of files/documents.

Reference is also made herein to patient data. As discussed in more detail below, the patient data includes information, both written text and visual images, that is entered by a patient, or her/his representative, into an active sheet medical questionnaire. The patient data is self-reported by a patient and provided in a structured manner through defined data fields of the active sheet. The patient data includes a main or chief complaint, one or more symptoms, and at least one of past medical history, past surgical history, current/past medications, current/past allergies, family and social history, history of present illness, or general activity level.

Reference is further made throughout to a clinical consultation note. As discussed herein, a clinical consultation note is an electronic form that provides a relevant, detailed medical history of a patient. The clinical consultation note includes certain sections configured to guide a physician through an examination of a patient. As such, the clinical consultation note includes sections for a review of patient symptoms, a diagnosis assessment to be completed by the physician, a principal plan to the completed by the physician, additional tests to be completed or selected by the physician, a discussion of risks/benefits to be completed by the physician, an impression of medical condition, or action items to be completed. The systems, methods, and apparatus disclosed herein are configured to enable at least some of these sections to be easily populated by a physician by selecting other sections with populated medical information or patient data. Further, the systems, methods, and apparatus are configured to use programmed logic to determine next actions based on content within the clinical consultation note. The next actions may include certain laboratory tests to be ordered, certain medical treatments to initiate (such as dialysis), a next appointment time period, general post-appointment timeline of predictable events, etc. Providing these next actions in a clinical consultation note enables a physician to effortlessly select which next actions are desired.

### I. Example Clinician Consultation Note System

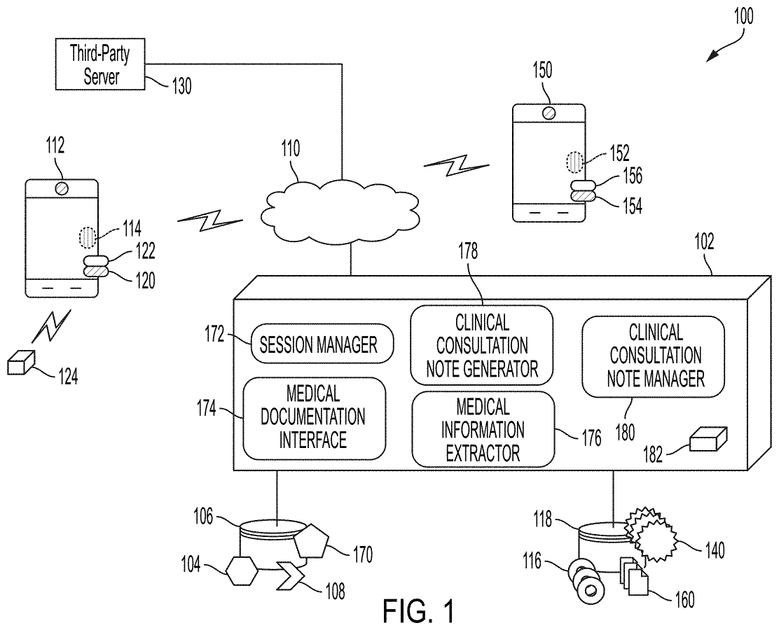

FIG. 1 illustrates a diagram of a clinical consultation note system 100, according to an example embodiment of the present disclosure. The example system 100 includes a management server 102 that is configured to manage one or more active sheet questionnaires 104 that are stored in a memory device 106. The management server 102, or the clinical consultation note system 100 generally may be incorporated into a medical network that has local access to patient EMRs, laboratory results, a pharmacy system, and medical testing/imaging systems.

As disclosed herein, the active sheet questionnaires 104 are configured to prompt patients for structured patient data. The active sheet questionnaires 104 include embedded logic or rules that specify more detailed medical questions based on initial patient data provided by a patient. The more detailed medical questions may be related to one or more possible medical conditions, such as a vascular or urologic issue.

The example memory device 106 may also include one or more clinical consultation templates 108 for creating clinical consultation notes. The templates 108 comprise a blank clinical consultation note with embedded programming that specify locations for certain structured patient data from the active sheet 104 and/or unstructured medical information from medical documentation. The memory device 106 may include random access memory (“RAM”), read only memory (“ROM”), flash memory, magnetic or optical disks, optical memory, or other storage media. While a single memory device 106 is shown, in other embodiments, the memory device 106 may be distributed within a cloud-computing network. In these examples, the active sheet questionnaires 104 may be provided through a distributed web-service.

The example management server 102 is communicatively coupled to a patient device 112 via a network 110. The patient device 112 includes any user device such as a smartphone, a tablet computer, a laptop computer, a desktop computer, a workstation, etc. The network 110 may include any wide-area network, such as the Internet, any cellular network, or combinations thereof While one patient device 112 is shown in FIG. 1, it should be appreciated that the management sever 102 may be communicatively coupled to hundreds or thousands of patient devices 112. The management server 102 is configured to host a separate session for each patient device 112 for guiding completion of a respective active sheet questionnaire 104.

The patient device 112 includes an application 114 that is configured to display and facilitate patient interaction with the active sheet questionnaire 104. For example, the application 114 may include one or more user interfaces with data fields that display data fields of the active sheet questionnaire 104. Patient inputs to the data fields are transmitted by the application 114 to the management server 102 as patient data 116, which is stored in a memory device 118. Similar to the memory device 106, the memory device 118 may include random access memory (“RAM”), read only memory (“ROM”), flash memory, magnetic or optical disks, optical memory, or other storage media.

In some embodiments, the application 114 is a web browser and the active sheet questionnaire 104 is a website or web form provided by the management server 102. In other embodiments, the application 114 is a software module or ‘app’ defined by one or more instructions 120 stored in a memory device of the patient device 112. A processor 122 of the patient device 112 is configured to execute the one or more instructions 120 to execute the active sheet questionnaire 104. In these embodiments, the data fields of the active sheet questionnaire 104 may be linked or mapped to one or more application programming interfaces (“APIs”) at the management server 102 for receiving the patient data 116 in a structured manner.

The example clinical consultation note system 100 of FIG. 1 also includes a third-party server 130. As disclosed herein, the active sheet questionnaire 104 enables a patient to either load their own medical documentation 140 or provide a name, address, or other identification information of a third-party to enable the management server 102 to obtain the medical documentation 140. In instances where a patient provides third-party information, the management server 102 is configured to request or otherwise access the medical documentation 140 from, for example, the third-party server 130. While FIG. 1 shows only one third-party server 130, other examples can includes tens to hundreds of third-party servers 130. The management server 102 uses the third-party information provided by a patient to select a third-party server 130. The request may include patient identification information, such as a name, social security number, patient number, address, etc. In response to the request, the third-party server 130 transmits medical documentation 140 that is related to the patient. This transmission also includes medical staff associated with the third-party server 130 uploading or faxing medical documentation 140 to the management server 102. The medical documentation 140 is generally unstructured and may include electronic files, test results, images, or scans of medical documents.

The example system 100 of FIG. 1 also includes one or more clinician devices 150, which may include any smartphone, tablet computer, laptop computer, desktop computer, workstation, etc. The clinician devices 150 are configured to display and enable completion of a clinical consultation note. The clinician device 150 may include an application 152 having one or more user interfaces for displaying the clinical consultation note. Clinician inputs to the clinical consultation note are transmitted by the application 152 to the management server 102 as, which is stored in the memory device 118 as part of a stored clinical consultation note 160.

In some embodiments, the application 152 is a web browser and the clinical consultation note 160 is a website or web form provided by the management server 102. In other embodiments, the application 152 is a software module or ‘app’ defined by one or more instructions 154 stored in a memory device of the clinician device 150. A processor 156 of the clinician device 150 is configured to execute the one or more instructions 154 to execute the clinical consultation note 160. In these embodiments, the data fields of the clinical consultation note 160 may be linked or mapped to one or more application programming interfaces (“APIs”) at the management server 102 for receiving the clinician inputs.

As illustrated in FIG. 1, the example management server 102 includes software modules 172 to 180 that are defined by instructions 170 stored in the memory device 106 (or another memory device). Execution of the instructions 170 by one or more processors 182 cause the management server 102 to perform the operations described herein. The processor 182 may comprise digital and/or analog circuitry structured as a microprocessor, application specific integrated circuit (“ASIC”), controller, etc.

The example software modules 172 to 180 are provided for illustration purposes only as partitions of operations performed by the instructions 170. In some embodiments, the management server 102 may include fewer or additional software modules 172 to 180. In other instances, at least some of the software modules 172 to 180 may be combined. In the illustrated example, the software modules include a session manager 172, a medical documentation interface 174, a medical information extractor 176, a clinical consultation note generator 178, and a clinical consultation note manager 180.

The example session manager 172 is configured to host or maintain a network session for each of the applications 114 on the patient devices 112. The session manager 172 may provide a log-in screen to authenticate a patient. The session manager 172 may also transmit a copy of the active sheet questionnaire 104 after receiving a request message from the patient device 112. In other instances, the session manager 172 may receive patient data 116 as structured data from defined data fields of the active sheet questionnaire 104, whether presented in a web browser application 114 or provided in a native application 114.

In some embodiments, the session manager 172 may apply patient responses to certain data fields as inputs to one or more embedded rules or logic of the active sheet questionnaire 104 to generate more detailed medical questions. Alternatively, the application 114 may apply the patient response to one or more rules for generating the more detailed medical questions. Additionally or alternatively, the active sheet questionnaire 104 is able to compare patient responses to one or more rules for generating additional data fields with the more detailed medical questions.

In some embodiments, the session manager 172 is configured to host a virtual session with the patient and a consultant. The virtual session may include a videoconferencing or teleconferencing platform where the consultant asks a patient questions from the active sheet questionnaire 104 or asks the patient to expand further upon previously entered information. In this manner, the consultant fills in or elaborates on the data fields for the patient. However, the active sheet questionnaire 104 still contains embedded logic that selects which more detailed medical questions are asked based on the patient data 116 input by the consultant. For video conferencing, the session manager 172 may display a video of the consultant split-screen with the data fields and interfaces of the active sheet questionnaire 104. The session manager 172 maybe configured to enable a patient's family members and/or physician to join the session to help provide the patient data 116 for the data fields of the active sheet questionnaire 104.

The session manager 172 may also include a natural language processing engine to transcribe patient responses. The natural language processing engine is configured to convert a patient's and/or consultant's speech to text and search for keyword triggers, such as “provider history” “provider exam”, etc. Identification of a trigger causes the engine to select the following text for inclusion with the corresponding data field of the active sheet questionnaire 104.

In some embodiments, the active sheet questionnaire 104 is configured to accept patient data 116 from one or more peripheral devices 124 that are communicatively coupled to the patient device 112. The peripheral devices 124 may include a fitness tracker, a smartwatch, a smart-ring, a wireless weight scale, a blood pressure cuff, etc. The peripheral devices 124 may be connected to the patient device 112 via a Bluetooth® connection, a Wi-Fi connection, or other wireless connection. In these embodiments, a patient may select to import or otherwise select patient data 116 from the peripheral devices 124 to complete one or more sections of the active sheet questionnaire 104. For instance, the patient may select (using the application 114 and/or the session manager 172) to import weight data from a weight scale, heart rate data from a fitness tracker, and sleep pattern data and counted steps from a smartwatch.

The session manager 172 outputs the patient data 116 recorded in the active sheet questionnaire 104 to the memory device 118. As discussed below, the patient data 116 constitutes structured data that is associated to one or more sections of the clinical consultation template 108. This enables the patient data 116 to be added to a clinical consultation note 160 directly from the entered patient data 116, thereby reducing any potential opportunities for data entry errors. In such a configuration, the active sheet questionnaire 104 is specifically configured for eliciting the patient data 116 from a patient in a format that is defined by the clinical consultation template 108.

As discussed in detail below, the active sheet questionnaire 104 may determine medical document needed based on the received patient data 116. Alternatively, the active sheet questionnaire 104 may generally ask a patient for medical documentation 140 and/or contact information of a third-party that possesses medical documentation 140. The medical documentation interface 174 is configured to provide for the transfer of the medical documentation files from the patient device 112 and/or the third-party server 130. The medical documentation interface 174 may host a file-transfer program that enables patients to copy medical documentation 140 from the memory device 120 of their patient device 112 into a file interface of the active sheet questionnaire 104. After the medical documentation 140 is specified via the application 114, the medical documentation interface 174 obtains or otherwise stores a copy of the medical documentation 140 to the memory device 118.

In instances where a third-party has the medical documentation 140, the medical documentation interface 174 transmits one or more request messages to the third-party server 130. The request messages may identify the medical documentation 140 needed, a return address of the medical documentation interface 174 and/or management server 102, and/or patient identification information. In response, the medical documentation interface 174 receives the medical documentation 140, which is stored to the memory device 118. It should be appreciated that the response may occur hours to days after the request in instances where medical staff have to manually locate and copy the medical documentation 140. As discussed below, the medical documentation 140 is generally unstructured with respect to the clinical consultation templates 108. Accordingly, the management server 102 is configured to perform an intermediate data processing step to extract relevant medical information from the medical documentation 140.

In some embodiments, the medical documentation interface 174 may also determine or receive an indication that at least some medical documentation 140 is internal to a medical provider associated with the management server 102. In such circumstances, the medical documentation interface 174 transmits a request to or otherwise accesses an EMR database. The medical documentation interface 174 may use a patient identifier to obtain one or more EMRs associated with the patient. The medical documentation interface 174 may also access the EMR database to obtain clinical notes, laboratory results, treatment history, medical history, medications, and/or any other medical-related information. Generally, even the medical documentation 140 from an EMR database may be unstructured with respect to the clinical consultation templates 108. However, in some instances, at least some of the medical documentation 140 may be structured and stored by the medical documentation interface 174 as the patient data 116 to the memory device 118.

The example medical information extractor 176 is configured to identify and/or extract relevant medical information from the medical documentation 140. For text-based files, the medical information extractor 176 is configured to perform keyword matching to search for the relevant medical information. The keyword matching may correspond to known keywords needed for population of the clinical consultation note 160, such as ‘laboratory results’, ‘weight’, ‘medications’, etc. Once keywords are identified, the medical information extractor 176 may copy the related medical information, including any images. The medical information extractor 176 may also analyze metadata or field identifiers as part of the matching.

For scans or images of documents, the medical information extractor 176 is configured to perform one or more optical character recognition (“OCR”) routines. The medical information extractor 176 is configured to convert images from the medical documentation 140 into text. The medical information extractor 176 may then perform keyword searching, as described above, for pre-specified information such as labs, pathology reports, and/or medical images. The medical information extractor 176 may also search for medical images, graphs, tables, etc.

The medical information extractor 176 may assign a data label or metadata to the extracted information. The data label or metadata provides an association with at least one section of the clinical consultation template 108. For example, the medical information extractor 176 may label an image as an ‘X-ray medical image’ or a series of numbers and text as “laboratory data”. The medical information extractor 176 may provide more specific labels based on the level of detail of text extracted.

In some embodiments, the medical information extractor 176 creates a link or association with the extracted medical information and a location in the medical documentation 140 from which the medical information was extracted. The link or association identifies the medical document 140 or file from which the medical information was extracted. The link or association may also identify a page number and/or page location of the medical document 140 or file from which the medical information was extracted. The medical information extractor 176 stores the link or association with the medical information in the memory device 118.

The example clinical consultation note generator 178 is configured to combine the extracted medical information from the medical documentation 140 with the patient data 116 in the clinical consultation template 108 to create the clinical consultation note 160. The template 108 defines one or more sections for receiving certain patient data 116 and extracted medical information. The clinical consultation note generator 178 uses the data labels or metadata of the medical information from the medical documentation 140 to determine appropriate sections of the template 108 for population. The clinical consultation note generator 178 may populate the patient data 116 using known correspondences between the patient data 116 and sections of the template 108. The clinical consultation note generator 178 may also add the links or associations to at least some of the medical information added to the template 108. The links may be displayed as colored and/or underlined words, selection of which causes the management server 102 to display the corresponding portion of the medical documentation 140. In other instances, the links may be displayed as selectable icons.

The clinical consultation note generator 178 may also provide color coding logic for certain sections of the clinical consultation note 160 that require a physician's attention. The logic may specify that certain fields or text of the clinical consultation note 160 are to be a certain color, such as red or yellow, until selected by the physician. This provides evidence that the physician reviewed the important medical information or patient data. The color coding also provides a directed physician workflow to ensure all relevant questions/information is discussed with the patient or otherwise considered during the consultation. The color-coding functionality may remove the coloring, change the coloring, etc. after review by the physician.

The clinical consultation note manager 180 is configured to provide for completion and processing of the clinical consultation note 160. After the note 160 is generated, the clinician device 150 may transmit a request message for the clinical consultation note. Alternatively, the clinical consultation note manager 180 may make the clinical consultation note 160 available on a dashboard or file repository associated with the physician and viewable by the application 152 on the clinician device 150.

As described below in more detail, the clinical consultation note manager 180 is configured to reduce a physician's time needed to complete the clinical consultation note 160 during and after a consultation with a patient. In some embodiments, the clinical consultation note 160 includes sections that have to be completed by a physician. The sections include a physician exam, a diagnosis/assessment, a principal plan, and additional test/next actions. The clinical consultation note manager 180 enables certain patient data and/or medical information already in the clinical consultation note 160 to be copied or otherwise selected by a physician for entry in the one or more sections. For example, if the physician wants to confirm a patient's physical exam, the clinical consultation note manager 180 enables a physician to select exam notes from the patient's medical history in the clinical consultation note 160 and write the selected exam notes in the selected section. Such a configuration reduces the amount of effort needed to complete the one or more sections of the clinical consultation note 160. In some embodiments, the clinical consultation note manager 180 operates in connection with the application 152 and/or embedded logic within the clinical consultation note 160 to provide a more automated approach at completing the sections.

The clinical consultation note manager 180 may also include a natural language processing engine to transcribe patient responses. The natural language processing engine is configured to convert a patient's and/or physician's speech to text and search for keyword triggers, such as “provider history” “provider exam”, etc. Identification of a trigger causes the engine to select the related text for inclusion with the corresponding section of the clinical consultation note 160.

In some embodiments, the clinical consultation note manager 180 is configured to host a teleconference for the patient consultation. In these embodiments, the clinical consultation note manager 180 provides an interface for the application 152 of the clinician device 150. One side of the interface shows a video chat session with a patient. Another side of the interface shows the clinician consultation note 160. Such a configuration enables a physician to populate the clinical consultation note 160 using the features provided by the clinical consultation note manager 180 while interacting with the patient virtually.

In some embodiments, the clinical consultation note manager 180 is configured to accept data from one or more peripheral devices that are communicatively coupled to the clinician device 150. The peripheral devices may include a peripheral device of the patient, such as the peripheral devices 124 or peripheral devices of a physician's office, which may include a heartrate monitor, a blood pressure cuff, a weight scale, a pulse ox meter, a glucose meter, a rapid blood or bodily fluid tester, etc. The peripheral devices may be connected to the clinician device 150 via a Bluetooth® connection, a Wi-Fi connection, or other wireless connection. In these embodiments, a physician may select to import or otherwise data from the peripheral devices to complete one or more sections of the clinical consultation note 160.

The clinical consultation note manager 180 may also provide for ensuring colored sections are viewed by a physician. The clinical consultation note manager 180 may analyze a clinical consultation note 160 when a physician attempts to finalize. The clinical consultation note manager 180 may prevent the clinical consultation note 160 from being finalized until all of the color coded sections are reviewed. In other embodiments, logic within the clinical consultation note 160 is configured to prevent a submit/finalization button from being active until all colored sections are reviewed by a physician.

After finalization, the clinical consultation note manager 180 is configured to store the clinical consultation note 160 to the memory device 118 and/or an EMR database. In addition, based on which next actions were selected by a physician in the clinical consultation note 160, the clinical consultation note manager 180 may cause the actions to be performed. This can include scheduling a new appointment, transmitting a medication prescription to a pharmacy, transmitting a consult note to a referring physician, automated ordering of further diagnostic tests or treatments, automated billing, automated creation of a post-discharge and/or home progress note, transmit home instructions to the patient device 112, transmit or assign education content for the patient, and/or handle post-discharge patient questionnaires and/or photo uploading. In some embodiments, the clinical consultation note manager 180 operates with a hospital system or EMR database to perform at least some of the next actions selected in the clinical consultation note 160.

In some embodiments, the clinical consultation note 160 and/or the patient data 112 received from a completed active sheet questionnaire 104 may be secured using a distributed ledge or blockchain framework. In these embodiments, the clinical consultation note 160 and/or the patient data 112 may be written in a distributed database to ensure the originality of the data and prevent the data from being overwritten or altered. The example management server 102 may use any blockchain or distributed ledger technology for securing the clinical consultation note 160 and/or the patient data 112.

### II. Example Clinician Consultation Note Process Flow Embodiment

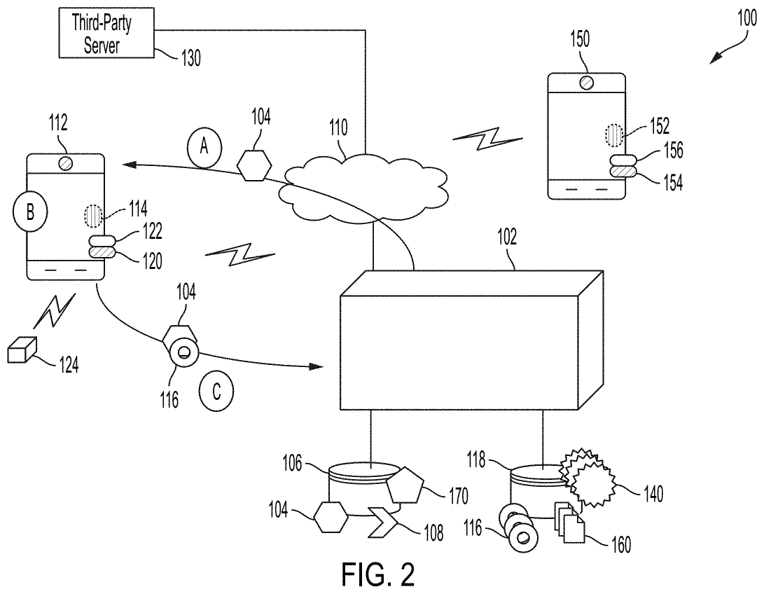

FIGS. 2 to 4 are diagrams that are illustrative of a process flow for creating a clinical consultation note 160, according to an example embodiment of the present disclosure. In FIG. 2, a patient of the patient device 112 uses the application 114 to access the management server 102. In response to the access, the session manager 172 of the management server 102 begins a new session and provides a blank active sheet questionnaire 104 to be completed (Event A). As discussed above, the active sheet questionnaire 104 includes logic and/or rules that define when certain data fields related to more detailed medical questions are to be displayed based on a patient's selection of symptoms and/or entry of other patient data 116.

The patient proceeds to populate data fields of the active sheet questionnaire 104 using the application 114 of the patient device 112 (Event B). In some instances, the session manager 172 of the management server 102 is configured to provide a teleconference session to assist a patient in completing the active sheet questionnaire 104. The active sheet questionnaire 104 is configured to record the patient data 116 via at least one of a Hypertext Transfer Protocol (“HTTP”) format, an HyperText Markup Language (“HTML”) format, an Extensible Markup Language (“XML”) format, a JavaScript Object Notation (“JSON”) payload, etc.). After the active sheet questionnaire 104 is completed, the application 114 transmits the active sheet questionnaire 104 with the patient data 116 to the management server 102 (Event C). In some embodiments, the application 114 may also provide medical documentation 140 provided by the patient.

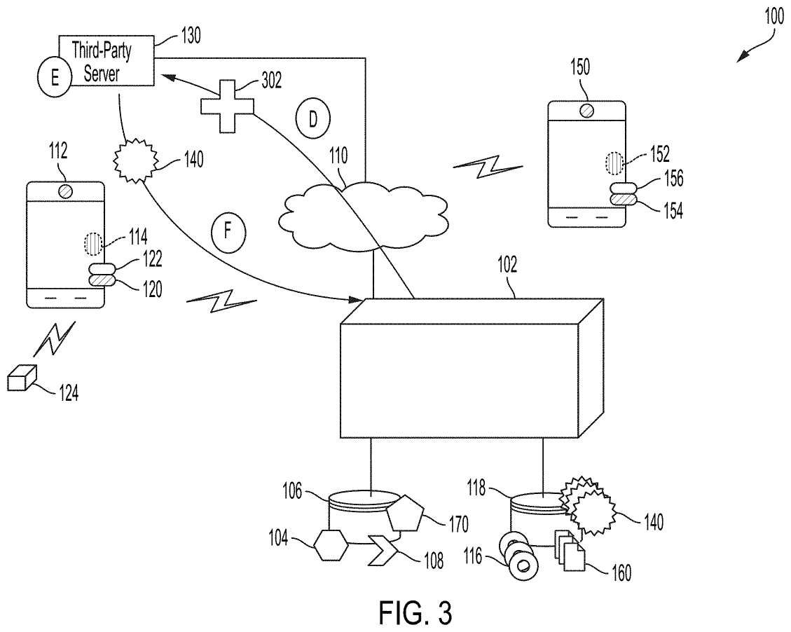

FIG. 3 shows an example where the patient specifies that at least some medical documentation 140 is located at the third-party server 130. Based on this information, the medical documentation interface 174 transmits a request message 302 to the third-party server 130 (Event D). The request message 302 includes an identifier of the patient, such as a name, serial number, social security number, etc. The request message 302 may also identify which medical documentation 140 is needed and/or provide a patient authorization that the medical documentation 140 can be released. At Event E, the third-party server 130 accesses or otherwise obtains the requested medical documentation 140. The third-party server 130 then transmits the medical documentation 140 in one or more messages to the management server 102 (Event F).

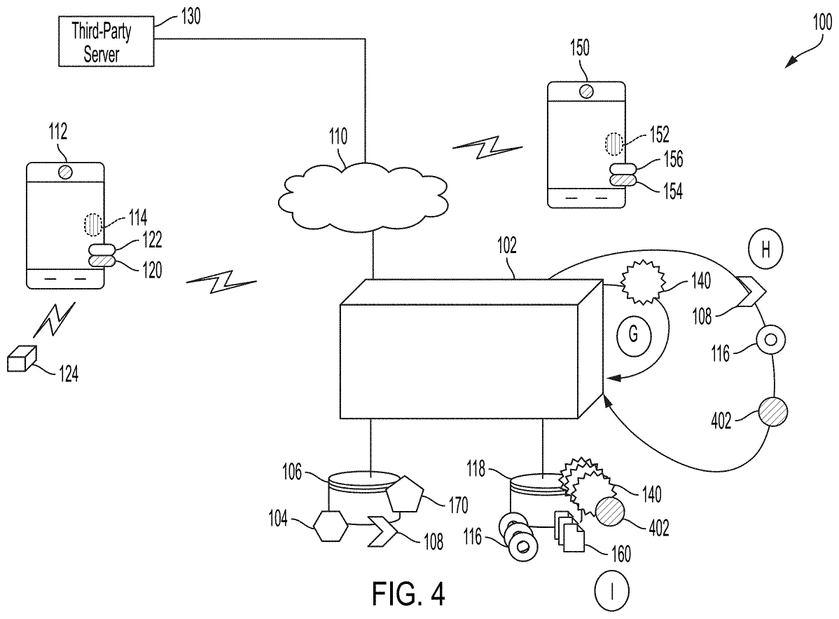

As shown in FIG. 4, the medical information extractor 176 is configured to analyze the medical documentation 140 to extract relevant medical information 402 (Event G). The medical information extractor 176 may also modify the extracted medical information 402 for readability. The extracted relevant medical information 402 is stored to the memory device 118.

At Event H, the clinical consultation note generator 178 is configured to apply the patient data 116 from the active sheet questionnaire 104 and/or the extracted relevant medical information 402 to the clinical consultation template 108 to generate the clinical consultation note 160. The management server 102 then stores the clinical consultation note 160 to the memory device 118. At this point, the clinical consultation note 160 is ready for completion by a physician after a consultation with a patient.

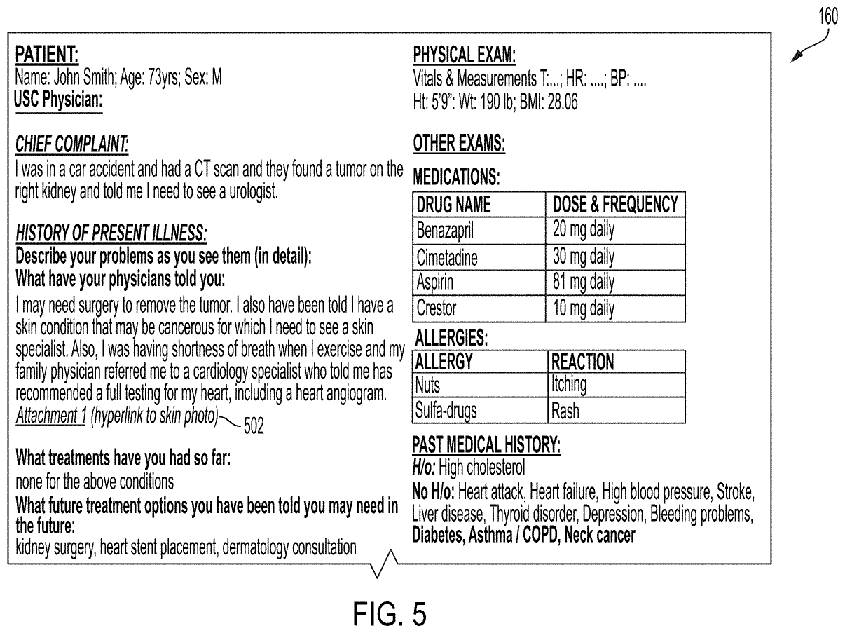

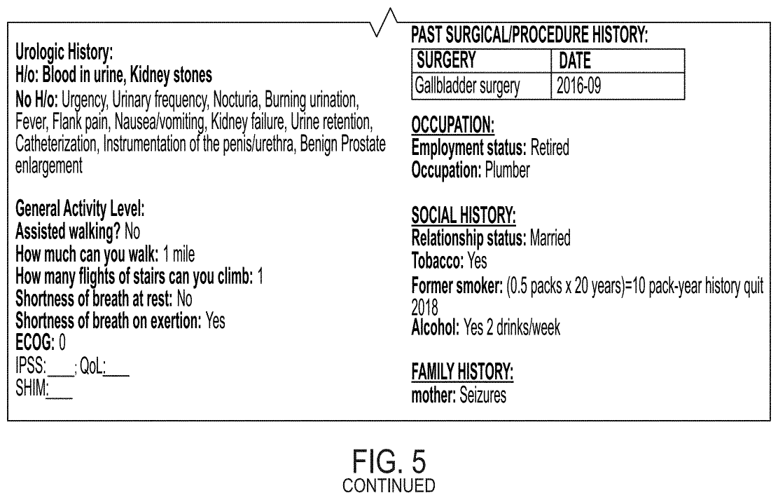

FIG. 5 shows a first portion of a clinical consultation note 160 after generation by the management server 102, according to an example embodiment of the present disclosure. As shown, the clinical consultation note 160 includes sections that are populated with the patient data 116. The template 108 may define a mapping between each of the sections and the structured patient data 116. The sections include Patient Name, Chief Complaint, History of Present Illness, Previous Treatments, Previous Medical History, General Activity Level, Physical Exam, Medications, Allergies, Past Surgical Procedures, Occupation, Social History, and Family History. In some embodiments, the Physical Exam patient data 116 may be obtained from the peripheral device 124. FIG. 5 also shows that the History of Present Illness section includes a link 502 to a photo uploaded by the patient. Selection of the link 502 by the physician causes the associated photo to be displayed in the application 152 of the clinician device 150.

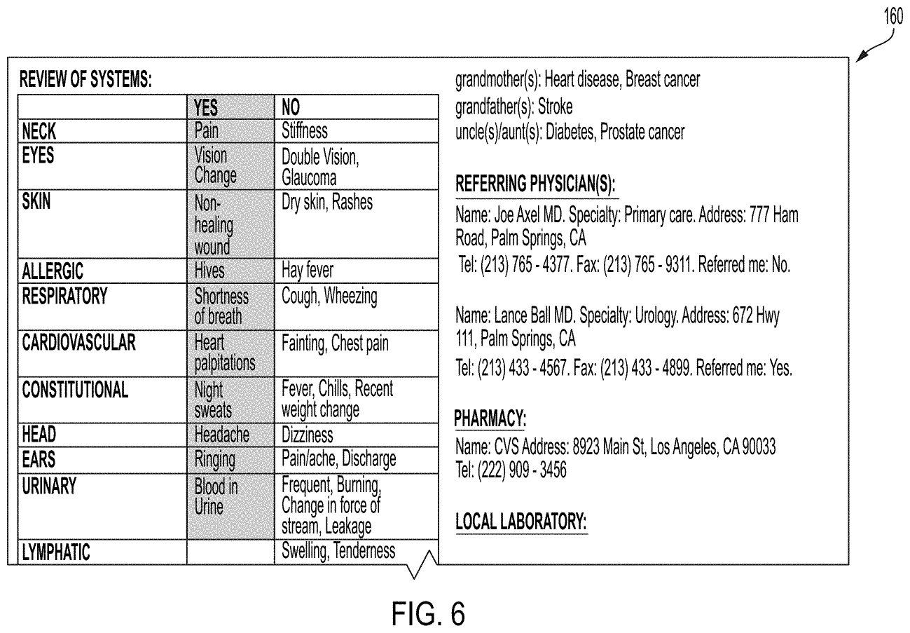

FIG. 6 shows a second portion of the clinical consultation note 160 after generation by the management server 102, according to an example embodiment of the present disclosure. The second portion of the clinical consultation note 160 includes a Review of Symptoms section, a Diagnostic/Assessment section, a Principal Plan section, Additional Tests Needed Section, a Referring Physician section, a Pharmacy Section, a Local Laboratory section, and a Test Done Till Date section. The Review of Symptoms section, the Referring Physician section, and the Pharmacy Section include structured patient data 116 from the active sheet questionnaire 104. The Local Laboratory section and the Test Done Till Date section include medical information 402 extracted from the medical documentation 140. Further, the Test Done Till Date section includes links, such as link 602, which specify a document location of the medical documentation 140. Selection of the link 602 causes the management server 102 and/or the application 152 to display the corresponding page or section of the source medical documentation 140.

FIG. 6 also shows shading for certain symptoms in the Review of Symptoms section of the second portion of the clinical consultation note 160. These symptoms are color coded to require a physician to select each symptom, which is indicative that the symptom was considered and/or discussed with a patient. After selection of the symptom the clinical consultation note 160 includes logic (or the management server 102) causes the text/box for the symptom to change color of have the color removed. The clinical consultation note 160 prevents a physician from completing the clinical consultation note 160 until all colored sections have been considered.

The Diagnostic/Assessment section, the Principal Plan section, and the Additional Tests Needed Section of the clinical consultation note 160 are to be populated by a physician during a consultation with a patient. As discussed above, any of the information shown in the clinical consultation note 160 may be selected by the physician for inclusion in these sections. Further, the clinical consultation note 160 is provided in a text form, which enables a physician to change, delete, or add to any of the included information.

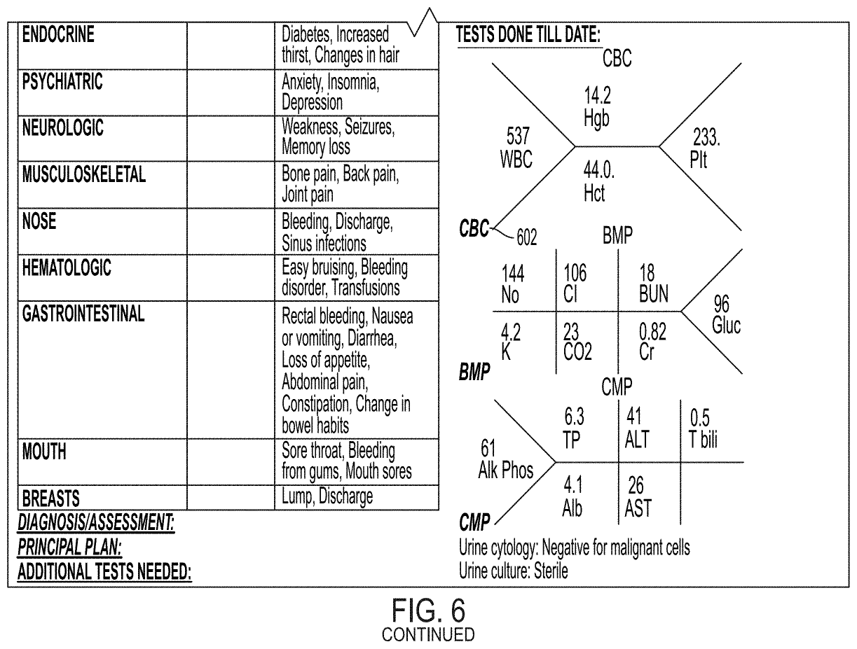

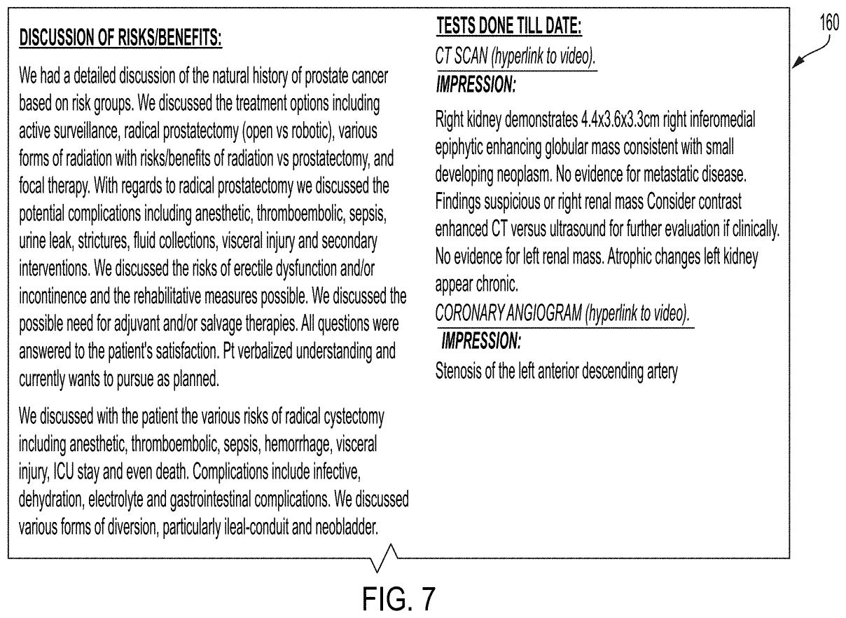

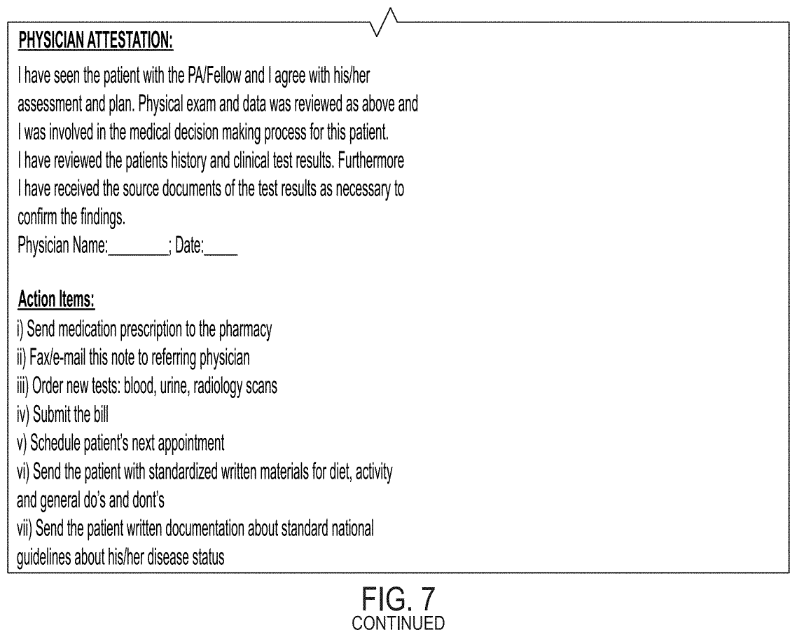

FIG. 7 shows a third portion of the clinical consultation note 160 after physician completion, according to an example embodiment of the present disclosure. In this example, the physician entered text for a Discussion of Risks section, a Physician Attestation section, an Impressions for the Tests Done till Date section, and An Action Item section. It should be appreciated that the management server 102 is configured to determine possible action items from the patient data 116 and/or the medical information 402. For example, selection of certain symptoms may be coded by one or more rules to a certain disease condition, which is associated with certain tests or actions. In this example, the management server 102 is configured to select the corresponding actions for inclusion in the clinical consultation note 160. A clinician may then select one or more of the action items, which cause the management server 102 (or hospital system) to automatically perform those actions when the clinical consultation note 160 is completed/finalized.

As shown above, the example management sever 102 enables the automated construction of a substantially complete patient clinical summary (e.g., the clinical consultation note 160) prior to the actual patient visit and prior to a physician/provider seeing and interacting with the patient for the first time. In this way, the management server 102 enables a physician to focus on the actual patient-physician interaction to make treatment decisions, provide treatment, counsel, educate, and make future treatment planning decisions, rather than perform routine data collection.

### II. Active Sheet Questionnaire Embodiment

The example active sheet questionnaire 104 includes a logic or algorithm driven, digital, detailed patient-intake form that is completed by a patient using the application 114 on the patient device 112 prior to seeing a physician/provider, either at home or in a clinic waiting room. The active sheet questionnaire 104 includes a plurality of data fields that may be provided on one or more pages. The management server 102 is programmed with a mapping of the data fields to sections of the clinical consultation note 160. For example, data fields for referring/primary care physician(s) and contact info, chief complaint, history of present illness, review of systems, past medical and surgical history, current medications, allergies, social history (e.g., occupation, marital status, smoking, drinking, drugs, etc.), and family history are mapped to corresponding sections in the clinical consultation note 160, as shown and described in connection with FIGS. 5 to 7. The direct mapping between the active sheet questionnaire 104 and sections of the clinical consultation note 160 enable the patient data 116 to be processed in a structured, known manner.

The active sheet questionnaire 104 may also be configured to directly interface (via the application 114 on the patient device 112) with various input sources (e.g., the peripheral devices 124) and/or external databases or APIs and smart devices (e.g., wearables, mobile phones). The patient data 116 is automatically extracted from external databases, EMRs or APIs and input into the active sheet questionnaire 104 using the management server 102 in cooperation with the application 114.

The active sheet questionnaire 104 includes embedded logic or algorithms that search for and identify keywords within the patient data 116 input by a patient (e.g., chief complaint, history of present illness, review of systems, medications, past medical/surgical history, etc.). Based on these keywords, logic or algorithms within the active sheet questionnaire 104 are configured to automatically present more detailed medical questions and/or determine specific test results (e.g., labs, radiology, pathology) that will be needed by a physician to make treatment decisions.

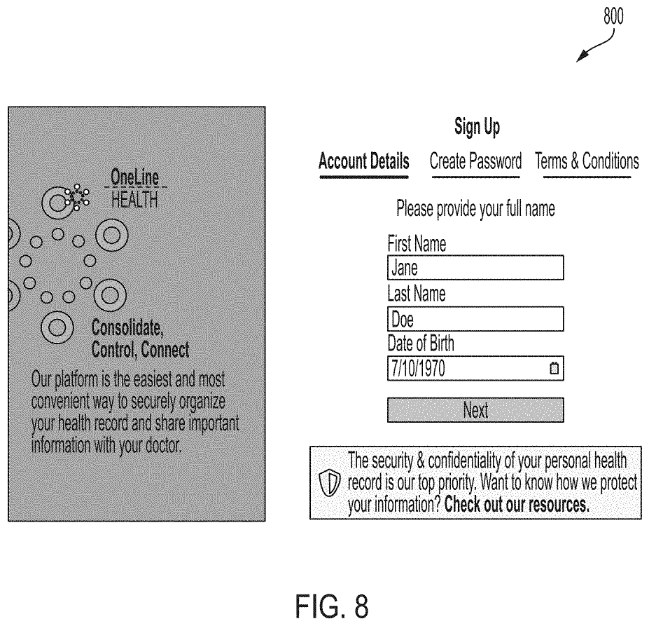

FIGS. 8 to 21 are diagrams that show different pages or user interfaces of the active sheet questionnaire 104, according to an example embodiment of the present disclosure. The user interfaces are displayed by the application 114 on the patient device 112. As shown in FIG. 8, a first step for completing an active sheet questionnaire 104 is patient registration. User interface 800 shows data fields for a patient to set up an account. The data fields include a name and a date of birth.

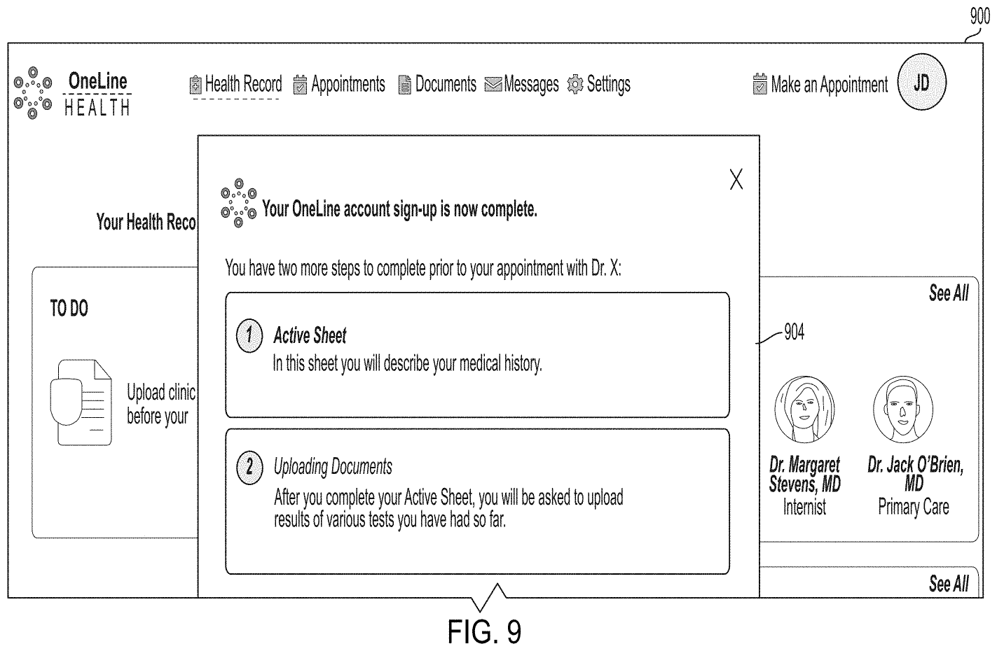

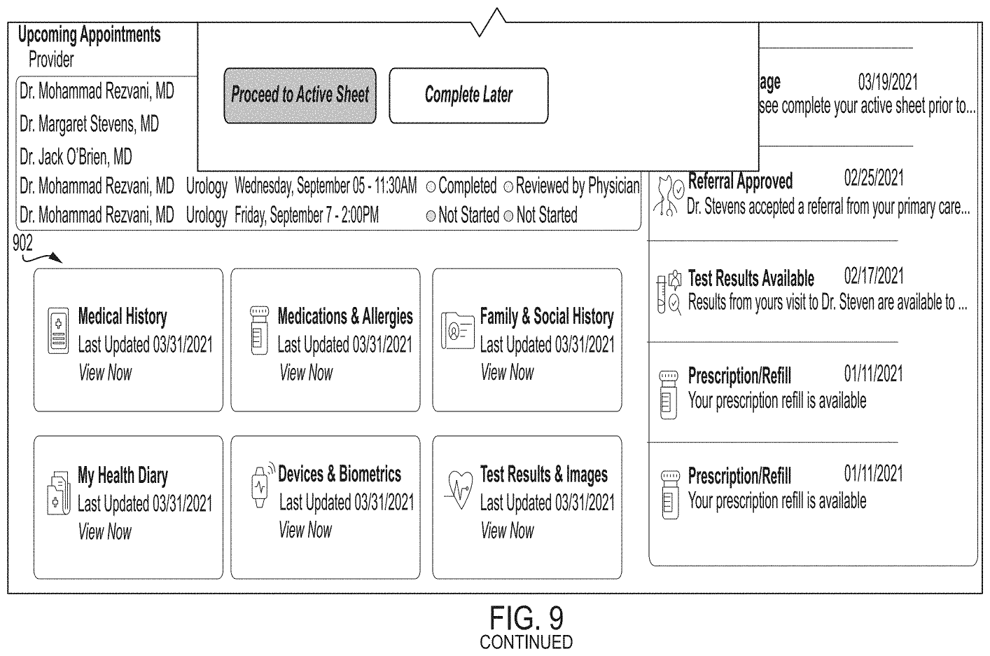

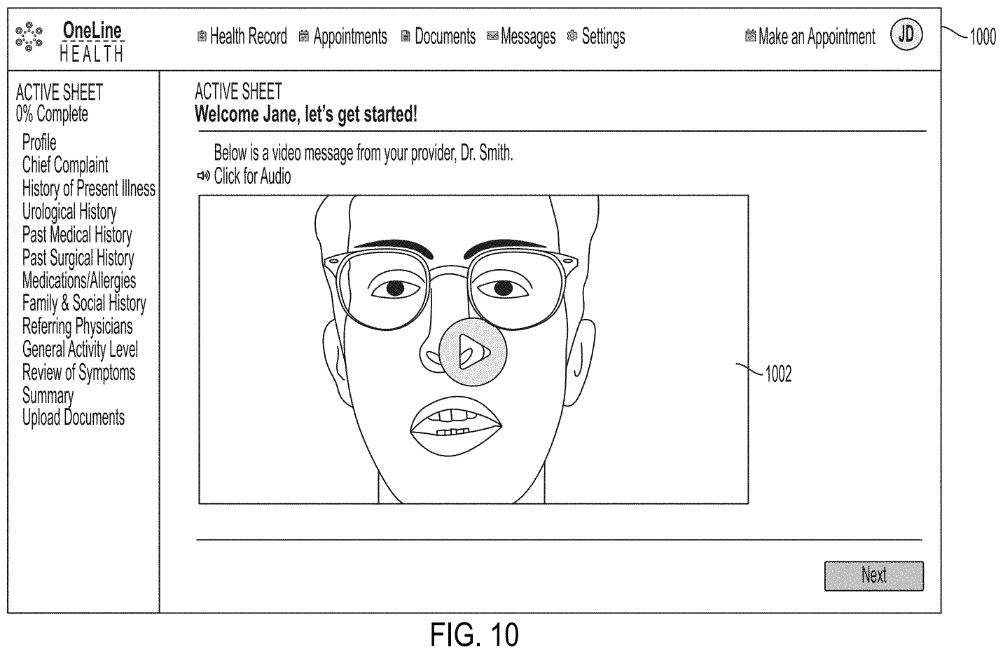

After registration, a user interface 900 is displayed to the patient, as shown in FIG. 9. The user interface 900 includes a patient dashboard 902 and a prompt 904 asking a patient to initiate a session to complete an active sheet questionnaire 104. The prompt 904 also provides an option for the patient to provide medical documentation 140. Selection of the option to start the active sheet questionnaire 104 causes a welcome user interface 1000 to be displayed, as shown in FIG. 10. In this embodiment, the user interface 1000 includes a video 1002 from a physician associated with the patient. The physician maybe a referring physician or a physician a patient has selected for a consultation. The video 1002 may provide encouragement for the patient and/or provide helpful advice for completing the active sheet questionnaire 104.

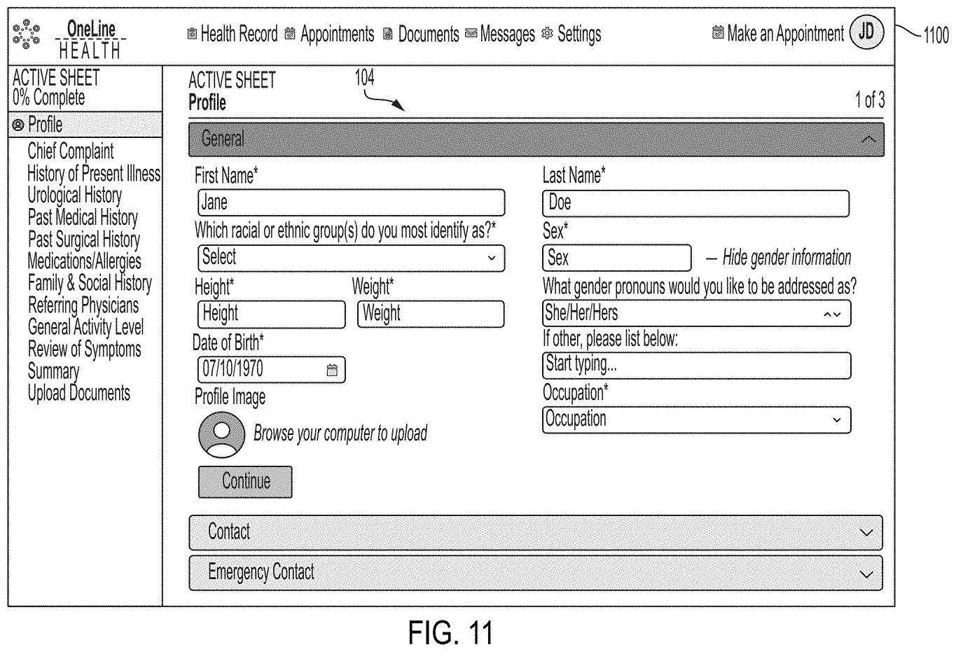

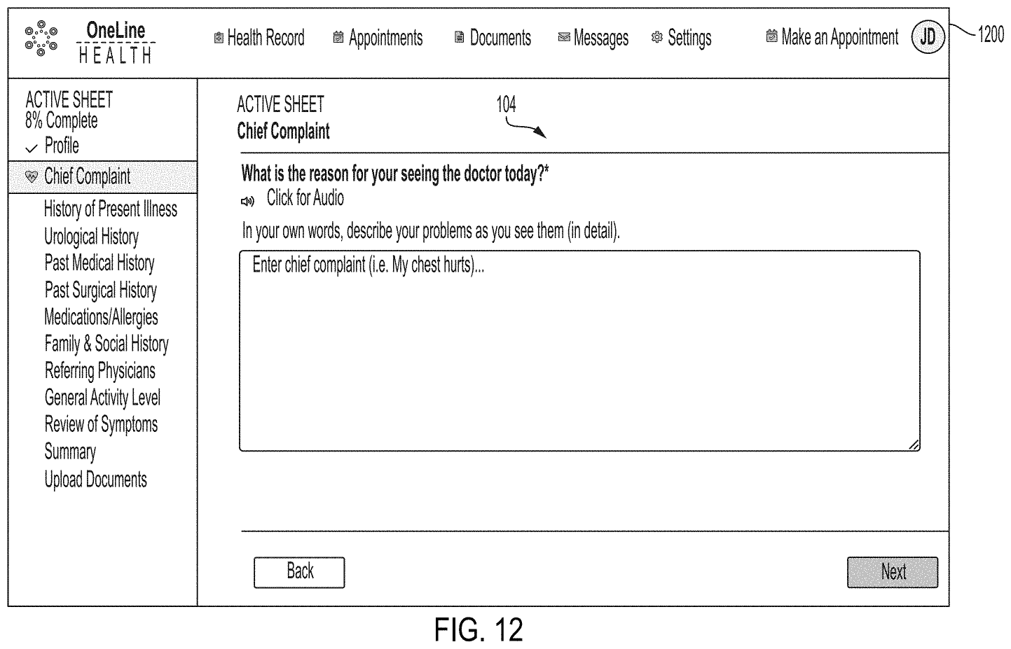

After the welcome user interface 1000, the application 114 and/or the management server 102 causes the active sheet questionnaire 104 to be displayed. FIG. 11 shows a patient profile user interface 1100 of the active sheet questionnaire 104. The patient profile user interface 1100 includes data fields for collecting patient data 116 including a patient name, height, weight, date of birth, sex, gender, and occupation. Further data fields may collect contact information and emergency contact information. FIG. 12 shows a chief complaint user interface 1200, which is provided after the profile user interface 1100. The chief complaint user interface 1200 includes a data field for recording patient data 116 related to a health problem that is afflicting a patient. The active sheet questionnaire 104 in cooperation with the management server 102 may use natural language processing to decipher the text and use keyword matching for identifying a possible medical condition.

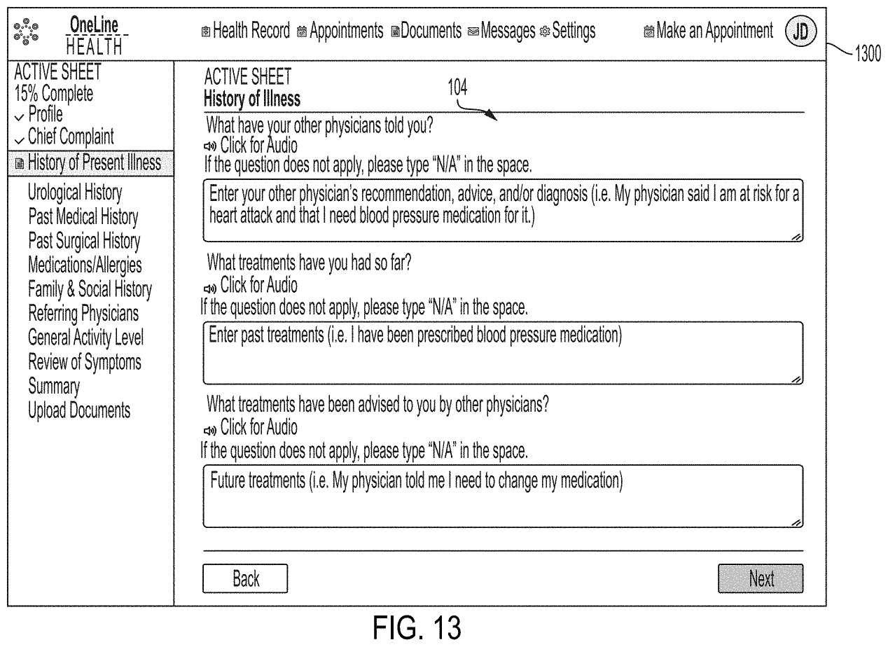

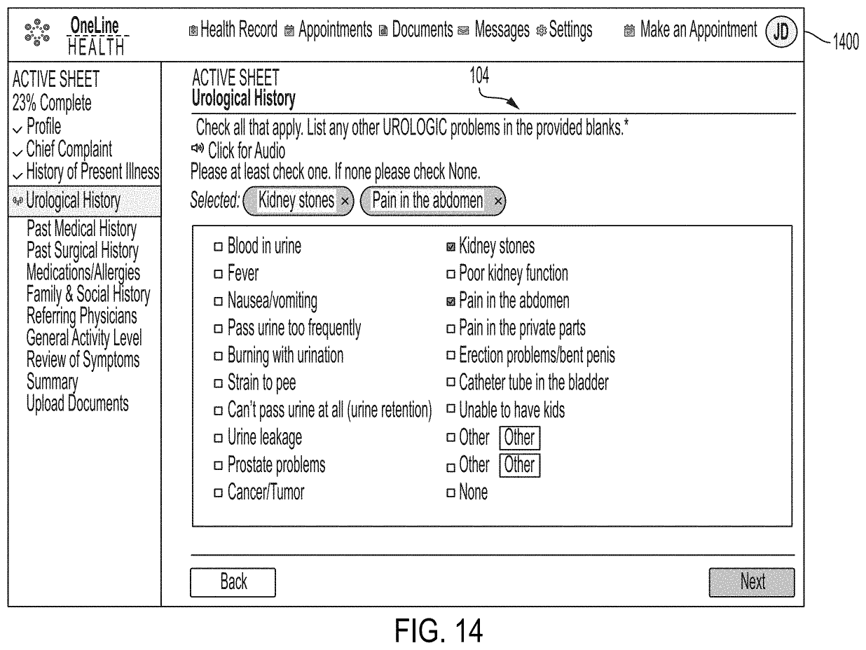

FIG. 13 shows a history of present illness user interface 1300 that is part of the active sheet questionnaire 104. The user interface 1300 includes data fields for recording past treatment information for the patient. FIG. 14 is a urological history user interface 1400. In this example, the patient's chief complaint and/or past medical treatments may include information indicative of a urologic medical issue. In this example, the patient has selected kidney stones and pain in the abdomen as symptoms. Other embodiments may be specific to all other medical or surgical sub-specialties such as cardiology, otolaryngology, endocrinology, etc.

Logic or an algorithm embedded within the active sheet questionnaire 104 (and/or the management server 102) uses keyword matching to determine the patient's medical condition is most likely urological. Based on this determination, the urological history user interface 1400 is displayed, which includes urologic symptoms for selection. In this manner, the user interface 1400 constitutes more detailed medical questions for answering by the patient. It should be appreciated that different user interfaces are provided based on an identified medical condition. For example, the user interface may include questions related to an International Prostate Symptom Score (“IPSS”) when keyword matching indicates the possibility of prostate cancer.

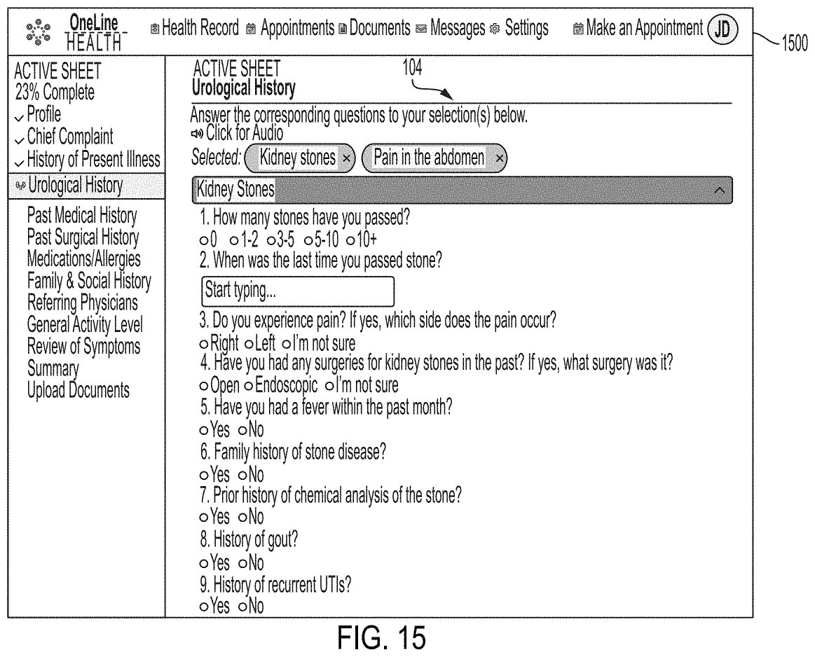

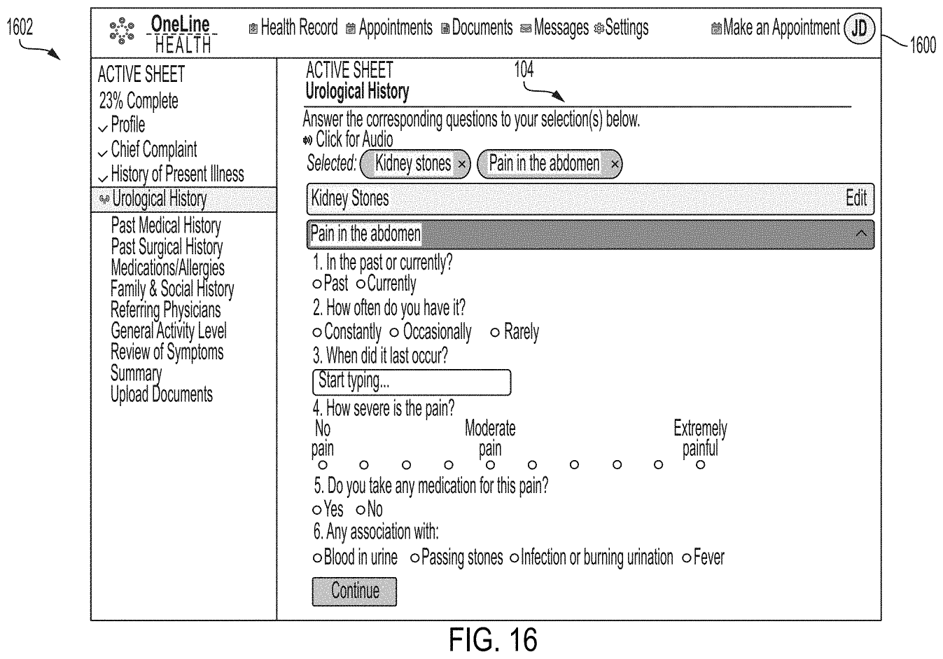

FIG. 15 shows a user interface 1500 that includes additional detailed medical questions that are displayed by the active sheet questionnaire 104 based on the patient selecting the kidney stones symptom. The user interface 1500 includes data fields that prompt a patient to provide more information about their kidney stones. FIG. 16 shows a user interface 1600 that includes additional detailed medical questions that are displayed by the active sheet questionnaire 104 based on the patient selecting the pain in the abdomen symptom. The user interface 1600 includes data fields that prompt a patient to provide more information about the pain in their abdomen.

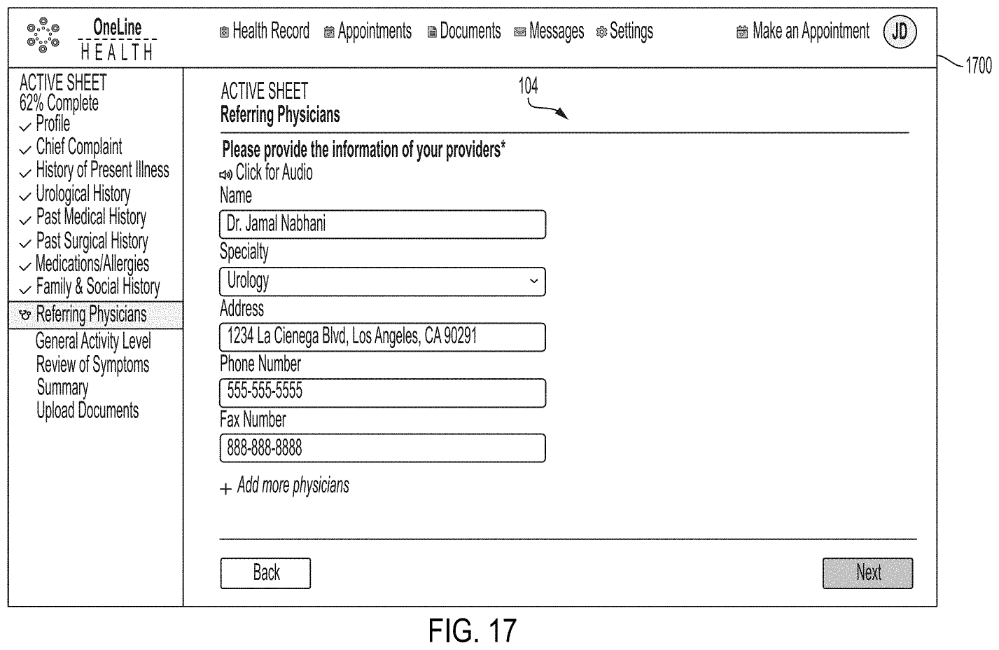

As shown in a progress bar 1602, the active sheet questionnaire 104 also includes user interfaces with data fields for obtaining patient data 116 related to a past medical history, a past surgical history, current medications/allergies, and a family/social history. For brevity, these user interfaces are not shown. User interface 1700 of FIG. 17 provides data fields for the active sheet questionnaire 104 regarding a referring physician. The management server 102 may use the data input into the user interface 1700 for transmitting request messages to the third-party servers 130 for medical documentation 140.

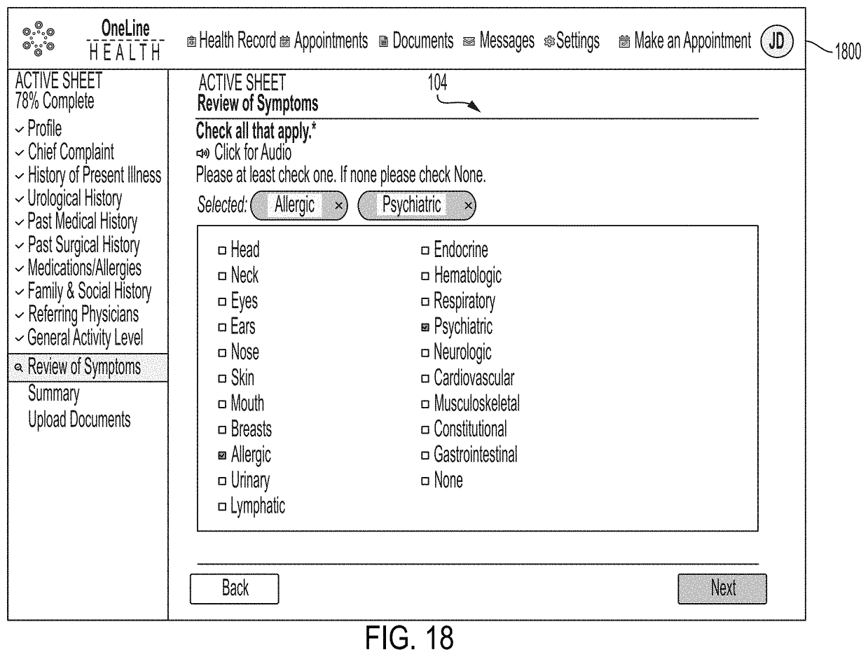

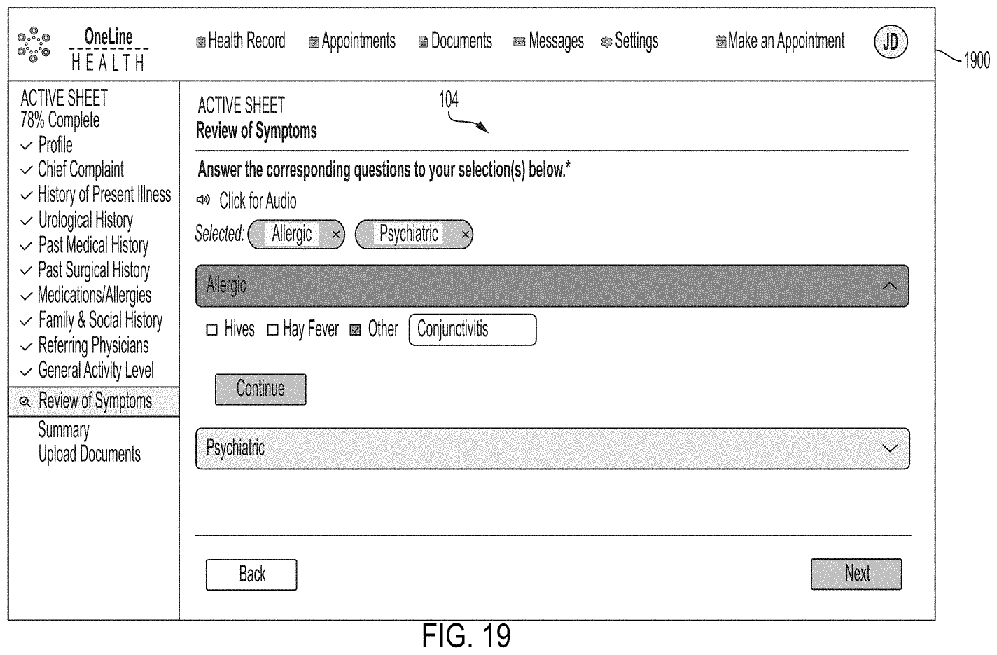

User interface 1800 of FIG. 18 provides data fields for the active sheet questionnaire 104 regarding further symptoms. In some embodiments, logic or an algorithm embedded within the active sheet questionnaire 104 (and/or the management server 102) uses keyword matching to determine which symptoms to display. The logic or an algorithm embedded within the active sheet questionnaire 104 may use patient data 116 entered into data fields of the previous user interfaces 1000 to 1700 for selecting which symptoms to show. In this example, a patient has selected allergies and psychiatric symptoms. As shown in the user interface 1900 of FIG. 19, the active sheet questionnaire 104 prompts with more detailed medical questions based on the selected symptoms.

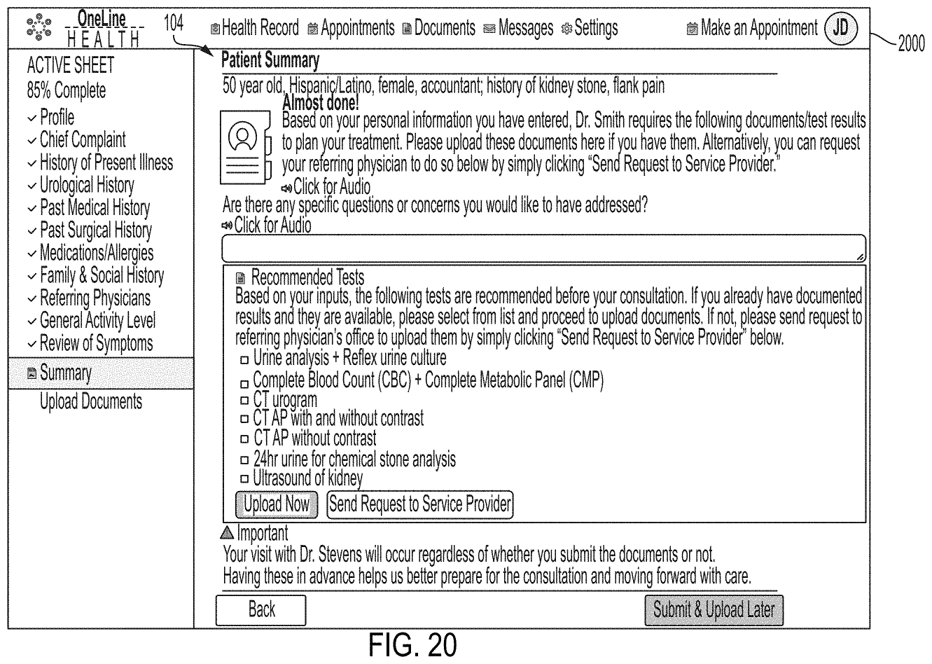

FIG. 20 shows a patient summary user interface 2000, according to an example embodiment of the present disclosure. In this example, logic or an algorithm embedded within the active sheet questionnaire 104 (and/or the management server 102) uses the previously entered patient data 116 to determine recommended medical tests for the patient. The tests may be defined by one or more rules that specify symptoms and other patient data needed to trigger the recommendation for a medical test. The active sheet questionnaire 104 uses embedded keywords to trigger algorithms or rules that connect that keyword with its partner diagnostic test and treatment plan, as recommended by national guidelines. For example, if the patient data 116 indicates “chest pain”, this keyword automatically triggers diagnostic test recommendations (e.g., a lipid profile, EKG, stress test, cardiology consult, etc.) and automatically places these tests into the user interface 2000. It will also automatically trigger the associated preliminary differential diagnoses (e.g., myocardial infarction vs Angina vs Reflux esophagitis, etc.) and attach a CMS diagnosis code. In so doing, the active sheet questionnaire 104 truly automates physician logic, reasoning, and critical thinking.

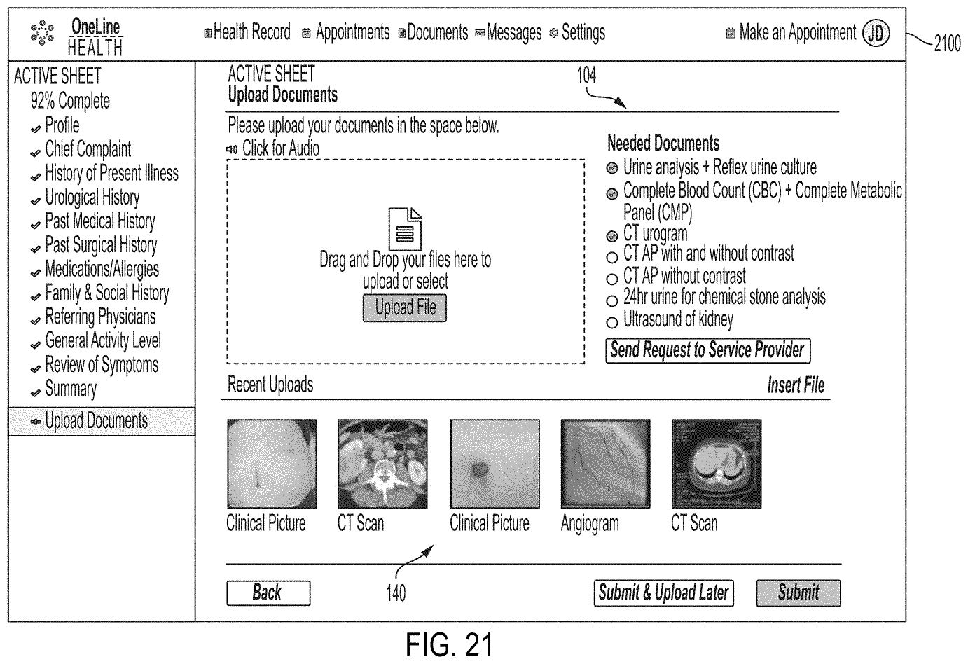

As shown in the user interface 2100 of FIG. 21, the active sheet questionnaire 104 includes data fields for a patient to provide medical documentation 140 for the identified test in the user interface 2000. In this manner, the active sheet questionnaire 104 enables a patient to provide unstructured medical documentation 140 to show that one or more recommended medical tests have already been performed. In this example, a patient has provided clinical pictures, CT scans, an angiogram, a urine analysis, and a complete blood count analysis. A patient may then hit the ‘submit’ button to complete the active sheet questionnaire 104.

If a patient is unable to provide the requested medical documentation 140, the management server 102 is configured to transmit request messages 302 to third-party servers 130 to obtain the medical documentation 140. If such medical documentation 140 is still not received by the consultation, the management server 102 is configured to add the medical tests to a list of recommended/next actions sections of the clinical consultation note 160, shown in FIG. 7.

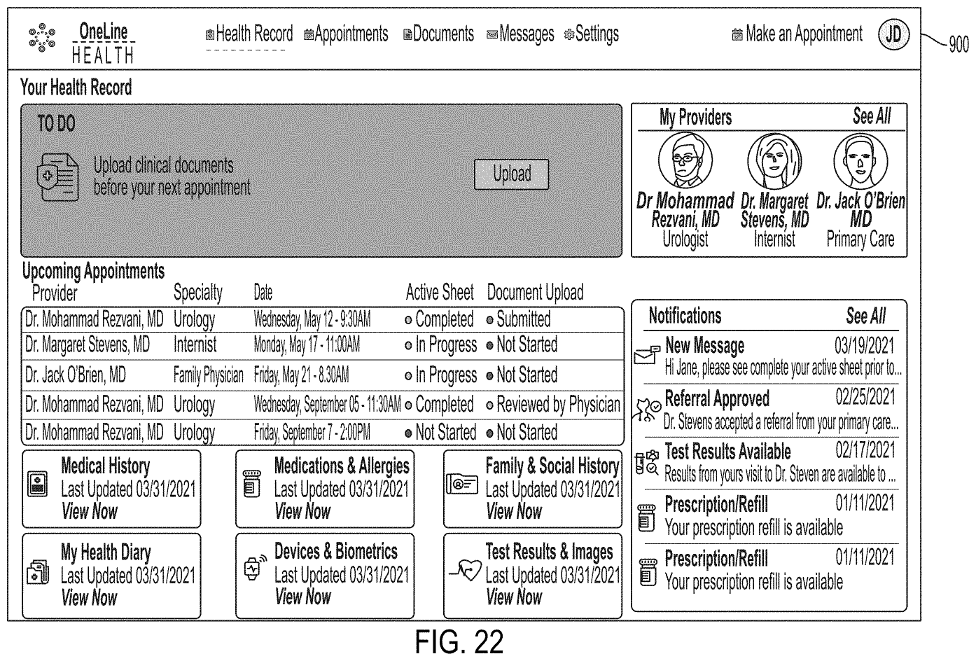

FIG. 22 is diagram of the dashboard 900 of FIG. 9 with the active sheet questionnaire 104 completed, according to an example embodiment of the present disclosure. The dashboard includes sections for upcoming appointments, with indications as to whether active sheet questionnaires 104 were completed for each appointment and whether medical documentation 140 was received. The dashboard 900 also includes options for a patient to view their medical history, medication/allergies, and other patient data 116 and/or medical documentation 140. The dashboard 900 also includes a feature to enable a patient to link or communicatively couple one or more peripheral devices 124 to their patient device 112 via a Devices and Biometrics option.

As shown above, the active sheet questionnaire 104 is programmed to ask every intricate, down-stream question that a physician would reasonably ask, thereby fully replicating the expert physician's thought processes and rationale, with stepwise questioning of increasing specificity. In other words, if a physician can think of a question, the active sheet questionnaire 104 is configured to include that question in one or more interfaces. Further, the active sheet questionnaire 104 is fully customizable, such that any individual physician can construct its detailed flow of questions according to that physician's personal practice and preference. Thus, the active sheet questionnaire 104 will not only ask the initial question about a medical symptom or ailment, but upon getting a positive answer, a drop-down menu or other data field is provided, which then asks all down-stream, associated, detailed ‘rabbit-hole’ medical questions, thereby obtaining and documenting all the intricate information that the physician would normally obtain in-person during a portion of the actual patient consultation. For example, the active sheet questionnaire 104 may ask an initial question, “Do you have chest pain?”. If the patient answers yes, then a drop-down menu of 15-20 additional “rabbit-hole” questions appear in the user interface or a subsequent user interface of the active sheet questionnaire 104 that ask all the related, detailed questions about chest pain, thereby obtaining the most complete clinical history. Doing so also serves to completely standardize the clinical history across an entire field, which is currently not possible. As alluded to above, patients are not subject to the individual skill level or knowledge base of each physician. Instead, the active sheet questionnaire 104 provides the highest level of clinical history from the patient to physicians rather than relying on individual physician performance to elicit all pertinent details.

Table 1 below provides an example of the logic or algorithms of the active sheet questionnaire 104 for a urology medical condition. It should be appreciated that the active sheet questionnaire 104 includes logic or algorithms for many or all possible medical conditions, whether physiological or mental. The table shows a first column for a selected patient symptom, which may have been selected in the user interface 1400 or the user interface 1800. The table also includes a column for more detailed medical questions for inclusion in subsequent user interfaces. Such questions are selected when a patient selects a corresponding symptom. A third column includes suggested or recommended medical testing. For example, for a kidney stones symptom, the logic of Table 1 specifies that recommended medical tests should include a urine analysis, a reflex urine culture, a complete blood count (“CBC”), a comprehensive metabolic panel (“CMP”), a 24-hour urine for stone profile, an ultrasound of a patient's kidney, and a CT of the patient's abdomen and pelvis without a contrast agent.

TABLE 1
Logic of an Active Sheet for a Urology Medical Condition

| | SUBSEQUENT QUESTIONING | |
|---|---|---|
| SYMPTOM | IF SYMPTOM IS SELECTED | SUGGESTED MEDICAL TESTING |
| Blood in | 1. What color is your urine? | 1. Cystosocpy |
| Urine | Answer choices (“AC”): bright, dark, rose | 2. CT Urogram |
| | 2. Do you have any pain? | 3. Urine cytology |
| | AC: scale of 1-10 | |
| | 3. Do you experience burning while urinating? | |
| | AC: Yes or no | |
| | 4. How often is there blood? | |
| | AC: always or occasional | |
| | 5. Associated with exercise or diet? | |
| | AC: yes or no | |
| | if yes, A.) then please explain in your own words. | |
| | 6. How many times have you experienced blood in the urine? | |
| | AC: 1-2, 3-5, 5-10, 10+ | |
| | 7. When does the blood occur during urination? | |
| | AC: at start, end or throughout urination? | |
| | 8. Do you have blood clots in the urine? | |
| | AC: Yes or no | |
| | 9. Have you had a fever within the past month? | |
| | AC: yes or no | |
| | 10. Have you had radiation therapy in the past? | |
| | (question repeated in past | |
| | AC: yes or no | |
| | 11. Are you a current/past smoker? (question repeated | |
| | in social history) | |
| | AC: yes or no | |
| | 12. Are you currently taking any blood thinners? | |
| | (ex. Aspirin, loganox . . . etc.) | |
| | AC: yes or no | |
| Kidney stones | 1. How many stones have you passed? | 1. Urine analysis, Reflex Urine Culture, |
| | AC: 0, 1-2, 3-5, 5-10, 10+ | 2. CBC, CMP |
| | 2. Fast time you passed stone? | 3. 24 hr urine for stone profile |
| | AC: free text | 4. Ultrasound of kidney |
| | 3. Do you experience pain? | 5. CT abdomen and pelvis without contrast |
| | If yes, which side does the pain occur? | |
| | AC: Right, Left, I'm not sure | |
| | 4. Have you had any surgeries for kidney stones in the past? | |
| | If yes, what surgery was it? | |
| | AC: Open, Endoscopic, I'm not sure | |
| | 5. Have you had a fever within the past month? | |
| | AC: yes or no | |
| | 6. Family history of stone disease? | |
| | AC: yes or no | |
| | 7. Prior history of chemical analysis of the stone? | |
| | AC: yes or no | |
| | 8. History of gout? | |
| | AC: yes or no | |
| | 9. History of recurrent UTIs? | |
| | AC: yes or no | |
| Urine | 1. How many time has this occurred till date times? | 1. Urine analysis, Reflex Urine Culture, |
| retention: | AC: 1, 2, 3+ | 2. CBC, CMP |
| Have you | 2. When was the first time? Last time? | 3. PSA (ONLY SUGGEST FOR MALE |
| been in a | 3. Did you require self-catheterization to pee? | PATIENTS*** WAIT MORE THAN 2 |
| situation | AC: yes or no | WEEKS FROM INFECTION/ |
| where you | | INSTRUMENTATION INCLUDING |
| could not pee | | CATHETER) |
| and needed a | | 4. Ultrasound of bladder and prostate |
| catheter to do | | |
| so? | | |
| Urgency | 1. When do you have urgency to urinate? | 1. Urine analysis, Reflex Urine Culture, |
| issues while | AC: Daytime, Nighttime, All the time | 2. CBC, CMP |
| urinating | 2. Do you ever leak with urgency? | |
| | AC: yes or no | |
| Fever within | 1. Any association with: | 1. Urine analysis, Reflex Urine Culture, |
| the past | AC: Blood in urine, Passing stones, Infection or burning urination | 2. CBC, CMP |
| month | | 3. Blood culture |
| | | 4. CT or MRI abdomen + pelvis |
| | | (with and without contrast) |
| Use of | n/a | 1. Urine analysis, Reflex Urine Culture, |
| catheter: | | 2. CBC, CMP |
| Have you | | 3. PSA (only suggest for male patients*** |
| ever had a | | wait more than 2 weeks from infection/ |
| urinary | | instrumentation including catheter) |
| catheter | | 4. Ultrasound of bladder and prostate |
| inserted? | | |
| Urinary | 1. How many times per day? | 1. Urine analysis, Reflex Urine |
| frequency | AC: 1-2, 3-5, 5-10, 10+ | Culture, |
| issues | 2. How many times per night? | 2. CBC, CMP |
| | AC: 1-2, 3-5, 5-10, 10+ | 3. Urodynamics |
| Flank pain | 1. In the past or currently? | 1. Urine analysis, Reflex Urine Culture, |
| | AC: Past, Currently | 2. CBC, CMP |
| | 2. How often do you have it? | 3. 24 hr urine for stone profile |
| | AC: constantly, occasionally, rarely | 4. Ultrasound of kidney |
| | 3. When did it last occur? | 5. CT or MRI abdomen + pelvis |
| | AC: free text | (with and without contrast) |
| | 4. How severe is the pain? | |
| | AC: scale of 1-10 | |
| | 5. Do you take any medication for this pain? | |
| | AC: yes or no | |
| | A.) if yes, what the medication is it (leave blank if unsure)? | |
| | 6. Any association with: | |
| | AC: Blood in urine, Passing stones, Infection or burning | |
| | urination, Fever | |
| Straining | 1. How often do you strain while urinating? | 1. Urine analysis, Reflex Urine Culture, |
| while | AC: Always or occasionally | 2. CBC, CMP |
| urinating | 2. Do you have a strong force of urinary stream? | 3. PSA (only suggest for male patients*** |
| | AC: yes or no | wait more than 2 weeks from infection/ |
| | 3. Can you empty your bladder to completion? | instrumentation including catheter) |
| | AC: yes or no | 4. Ultrasound of bladder and prostate |
| | 4. Do you stand at the toilet a little longer after you are | 5. Uroflowmetry |
| | done peeing to try and get more out? | 6. Post-void residual |
| | AC: yes or no | |
| | 5. Do you dribble urine? | |
| | AC: occasionally, often, only after finishing urination | |
| Nausea or | 1. Are you experiencing this symptom currently or in the past? | n/a |
| vomiting | AC: Currently or in the past? | |
| | 2. Last time you vomited? | |
| | AC: free text | |
| | 3. Associations with vomiting: | |
| | AC: Any blood? Any abdominal pain? | |
| | Any flank pain? Any food? | |
| Enlarged | 1. Have you had surgery for your enlarged prostate? | 1. Urine analysis, Reflex Urine Culture, |
| prostate | AC: Yes or no | 2. CBC, CMP |
| | If yes, A.) what surgery? (question repeated in past | 3. PSA (only suggest for male patients*** |
| | surgical history) | wait more than 2 weeks from infection/ |
| | B.) When was your surgery? (question repeated in past | instrumentation including catheter) |
| | surgical history) | 4. Ultrasound of bladder and prostate |
| | 2. Have you been told you may need surgery for it in the future? | 5. Uroflowmetry |
| | 3. Do you take any medications for it? | 6. Post-void residual |
| Burning | 1. How often do you experience burning while urinating? | 1. Urine analysis, Reflex Urine Culture, |
| urination | AC: Always, occasionally | 2. CBC, CMP |
| | 2. Is it associated with any of the following: | 3. Blood culture |
| | AC: blood in the urine? fever? | 4. CT or MRI abdomen + pelvis |
| | 3. Have you had any urine culture tests come back positive for | (with and without contrast) |
| | infection? | |
| | AC: yes or no | |
| | If yes: a. Do you know the name of the bug/bacteria | |
| | b. If treated, do you know the name of the antibiotic? | |
| | 4. Cloudy urine? | |
| | AC: yes or no | |
| | 5. Malodorous urine? (Foul smelling) | |
| | AC: yes or no | |
| Kidney | 1. Do you currently have dialysis | 1. Urine analysis, Reflex Urine Culture, |
| failure | AC: yes or no | 2. CBC, CMP |
| | If yes: A.) Hemodialysis? B.) Peritoneal dialysis? | 3. Ultrasound of kidney, bladder, prostate |
| | 2. Have you ever had dialysis in the past? | 4. Nephrology consult |
| | AC: yes or no | |
| | 3. Have you ever been told you may need dialysis in the future? | |
| | AC: yes or no | |
| | 4. Do you have a kidney doctor? | |
| | AC: yes or no | |
| | If yes: A.) Name of doctor | |
| Urine leakage | 1. Do you wear pads for urine leakage? | 1. Urine analysis, Reflex Urine Culture, |
| | AC: yes or no | 2. CBC, CMP |
| | If yes: A.) How many pads per day? | 3. Voiding diary |
| | AC: 0, 1-2, 3-4. 5+ | 4. Urodynamics |
| | B.) How wet are the pads | 5. Vaginal exam |
| | AC: little wet, quite wet, soaked | |
| | C.) What kind of pads do you use | |
| | AC: Small pads, maxipads, diapers, other | |
| | 2. How would you describe your urine leakage? | |
| | AC: Constant leakage? | |
| | Leak only when straining? (laugh, cough, sneeze, | |
| | exercise, etc.), | |
| | Leak only when you have an urge to pee? | |
| | 3. How long have you had leakage? | |
| | AC: days, weeks, months, years) | |
| Urinary tract | 1. Have you had any urine culture tests come back positive for | 1. Urine culture |
| infection | infection? | 2. Renal Ultrasound |
| (UTI) | AC: yes or no | |
| | If yes: a. Do you know the name of the bug/bacteria | |
| | b. If treated, do you know the name of the antibiotic? | |
| | 2. Would you describe your urine as cloudy? | |
| | AC: yes or no | |
| | 3. Would you describe your urine as having a foul smell? | |
| | AC: yes or no | |
| | 4. Do you experience blood in the urine? | |
| | AC: yes or no | |
| | ****Only had blood in urine with infection? | |
| | (IF YES, THEN RECHECK URINE ANALYSIS) | |
| Urologic | 1. Kidney Cancer | |
| Cancer(s) | AC: yes or no | |
| | If yes: a.) Which kidney? (R, L, Both, Don't know) | |
| | AC: Right, Left, I'm not sure | |
| | b.) How much of the kidney was removed | |
| | AC: Partial removal, total removal, I'm not sure | |
| | c.) Date of surgery + Surgeon | |
| | AC: free text similar to entering past surgical history | |
| Urologic | 2. Bladder Cancer | |
| Cancer(s) | AC: yes or no | |
| | If yes: a.) Was the bladder removed? (radical cystectomy) | |
| | AC: yes or no | |
| | b.) Did you receive chemotherapy? | |
| | AC: yes or no | |
| | c.) Date of surgery + Surgeon | |
| | AC: free text similar to entering past surgical history | |
| Urologic | 3. Adrenal Cancer | |
| Cancer(s) | AC: yes or no | |
| | If yes: a.) Which side? | |
| | AC: Right, Left, I'm not sure | |
| | b.) How much of the kidney was removed | |
| | AC: Partial removal, total removal, I'm not sure | |
| | c.) Date of surgery + Surgeon | |
| | AC: free text similar to entering past surgical history | |
| Urologic | 4. Prostate Cancer | |
| Cancer(s) | AC: yes or no | |
| | If yes: a.) Was the bladder removed? (radical cystectomy) | |
| | AC: yes or no | |
| | b.) Did you receive radiation therapy? | |
| | AC: yes or no | |
| | If yes, 1.)Date of surgery + Surgeon | |
| | AC: free text similar to entering past surgical history | |
| | C.) Did you receive HIFU or Cryosurgery? | |
| | AC: yes or no | |
| | d.) Did you receive any hormonal treatment? | |
| | AC: yes or no | |
| | e.) Did you receive any chemotherapy? | |
| | AC: yes or no | |
| | f.) Date of surgery + Surgeon | |
| | AC: free text similar to entering past surgical history | |
| Urologic | 5. Cancer of the penis | |
| Cancer(s) | AC: yes or no | |
| | If yes: a.) How much of the penis was removed? | |
| | AC: Partial removal, total removal, I'm not sure | |
| | b.) Did you receive radiation therapy? | |
| | AC: yes or no | |
| | If yes, 1.)Date of surgery + Surgeon | |
| | AC: free text similar to entering past surgical history | |
| | C.) Date of surgery + Surgeon | |
| | AC: free text similar to entering past surgical history | |
| Urologic | 6. Testes AC: yes or no | |
| Cancer(s) | If yes: a.) Which testes was affected with cancer? | |
| | AC: Right, Left, Both, I'm not sure | |
| | b.) How much of the testes was removed? | |
| | AC: Partial removal, total removal, I'm not sure | |
| | c.) Did you have lymph nodes removed from the abdomen? | |
| | (RPLND) | |
| | AC: yes or no | |
| | d.) Date of surgery + Surgeon | |
| | AC: free text similar to entering past surgical history | |

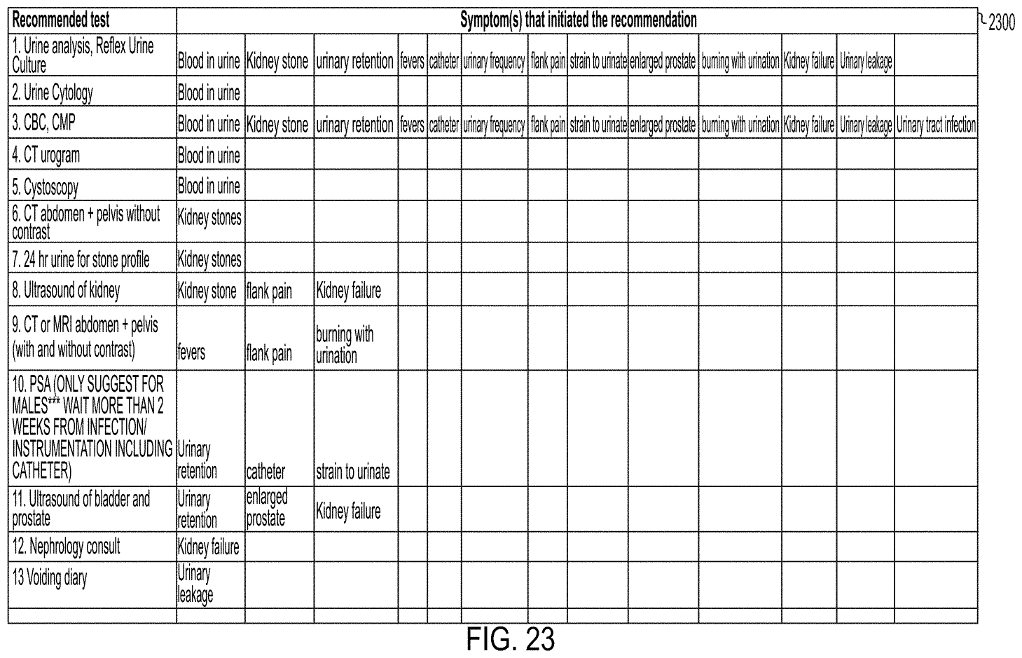

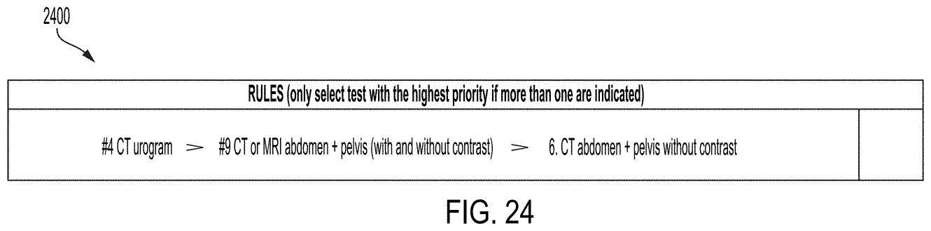

FIG. 23 is a diagram of a table 2300 that correlates recommended medical tests to symptoms for the logic or algorithms of the active sheet questionnaire 104, according to an example embodiment of the present disclosure. Further, FIG. 24 shows a rule 2400 for selecting a medical test, according to an example embodiment of the present disclosure. As shown in FIG. 23, a symptom may correspond to one or more medical tests, which may vary in complexity and data analyzed. A more complex test may provide the same information as a simple test, with the benefit of additional information. As such, the rule 2400 specifies that only the most complex matching medical test is recommended, thereby preventing unneeded medical test from being recommended.

In an example, a patient via the user interface 1400 of the active sheet questionnaire 104 selects symptoms of blood in their urine, burning urination, and kidney stones. Such symptoms correspond to a CT of the abdomen and pelvis, a CT or MIRI of the abdomen and pelvis, and a CT urogram. The rule 2400 specifies that the CT urogram covers the other tests and has a higher priority. As such, the active sheet questionnaire 104 returns a CT urogram as a recommended test for the user interface 2100 of FIG. 21.

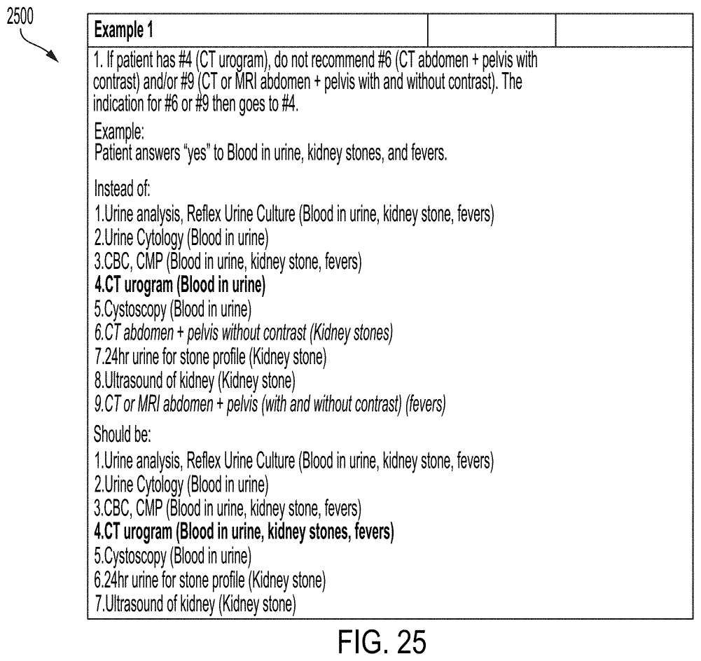

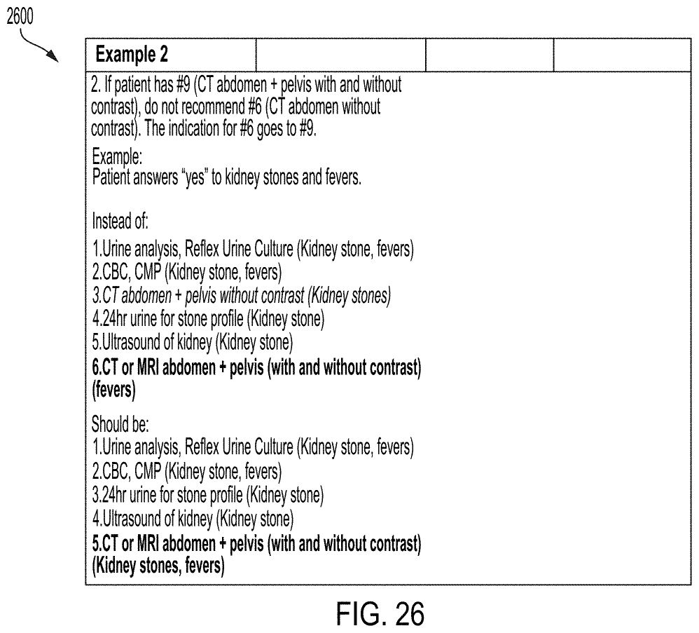

FIGS. 25 and 26 are diagrams that illustrate the logic of the active sheet questionnaire 104, according to example embodiments of the present disclosure. In FIG. 25, the active sheet questionnaire 104 uses the rule 2400 to determine that based on a patient's symptoms the following actions should be recommended: urine analysis, reflex urine culture, urine cytology, CBC, CMP, CT urogram, cystoscopy, a 24-hour urine stone profile, and an ultrasound of the patient's kidneys. As shown in FIG. 26, different symptoms cause the active sheet questionnaire 104 to use the rule 2400 to determine that the following actions should be recommended: urine analysis, reflex urine culture, urine cytology, CBC, CMP, a 24-hour urine stone profile, an ultrasound of the patient's kidneys, and a CT or MRI of the patient's abdomen and pelvis. The example management server 102 using the active sheet questionnaire 104 is accordingly configured to determine recommended medical tests or treatments for a patient using input patient data 116, thereby reducing the burden on physicians and eliminating potential oversights in treatment.

### IV. Clinical Consultation Note Generation Embodiment

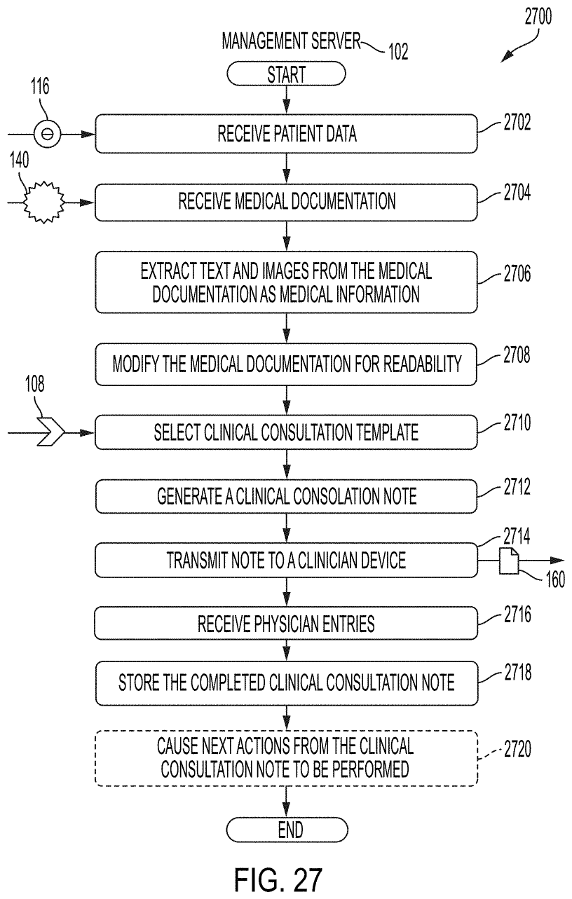

FIG. 27 is a flow diagram of an example procedure to create a clinical consultation note 160 using structured patient data and unstructured medical documentation, according to an example embodiment of the present disclosure. Although the procedure 2700 is described with reference to the flow diagram illustrated in FIG. 27, it should be appreciated that many other methods of performing the steps associated with the procedure 2700 may be used. For example, the order of many of the blocks may be changed, certain blocks may be combined with other blocks, and many of the blocks described may be optional. In an embodiment, the number of blocks may be changed based on use of the clinical consultation note 160. The actions described in the procedure 2700 are specified by one or more instructions and may be performed among multiple devices including, for example the management server 102 and/or the application 152 on the clinician device 150.

The example procedure 2700 begins when the management server 102 receives patient data 116 for a patient via an active sheet questionnaire 104 (block 2702). The management server 102 also receives medical documentation 140 via the active sheet questionnaire 104, as discussed above in connection with FIG. 21, from the third-party server 130 after a request message is transmitted, or from an internal medical system (block 2704). The management server 102 next extracts, as medical information 402, relevant text and/or images from the medical documentation 140 (block 2706). The management server 102 may use database that specifies which medical information 402 is needed using, for example, the patient data 116. In the example above, the patient data 116 may be indicative of a urology issue. The management server 102 accordingly uses a database that specifies keywords related to urology in the extracted text and images from the medical documentation 140. This may include searching for keywords of ‘urine’ and ‘test’ in text from the medical documentation 140 and copying the related or surrounding text. The management server 102 may also modify or otherwise amend the medical information 402 to improve readability or inclusion in a clinical consultation note 160 (block 2708).

The example procedure 2700 continues when the management server 102 selects a clinical consultation template 108 (block 2710). It should be appreciated that different clinical consultation templates 108 may be available. The management server 102 is configured to select the template 108 based on an identified medical condition from the patient data 116 and/or the medical information 402 from the medical documentation 140. For the example above, the management server 102 selects a urology template 108.

The management server 102 then uses the selected template 108 to create a clinical consultation note 160 (block 2712). As discussed above, a clinical consultation template 108 includes blank sections. A database or the template itself 108 may include logic that links the section to structured patient data 116 and/or unstructured medical information 402 from the medical documentation. The structured patient data 116 may be linked via direct mapping while the management server 102 may use keyword matching to determine which of the medical information 402 is to be included in the different sections of the template 108. The management server 102 uses the defined associations and keyword matching to populate at least a portion of the patient data 116 and/or the medical information 402 into the selected template 108, as discussed above in connection with FIGS. 5 to 7.

The management server 102 also creates associations between the medical information 402 stored in the note 160 and corresponding locations of the medical documentation 140. The created associations are shown as user-selectable links 602 in the clinical consultation note 160. By clicking on a hyperlink 602, a new window opens with the entire scanned/faxed medical documentation 140 available for viewing in two panes of the application 152: 1) the original image (usually PDF document) on which the OCR is based, which is displayed side-by-side with; 2) the searchable text file created by OCR. This serves two functions. First, it allows the physician to confirm the accuracy of the OCR results by viewing the OCR results side-by-side with the original source document. Second, it enables the provider to search, copy, and paste any additional text from the CT scan report (e.g., the medical documentation 140) that she/he wishes to include in the clinical consultation note 160. Further, another hyper-link connects with the EMR to enable the physician to immediately view the actual CT scan images of that patient.

The management server 102 may further provide color coding for certain sections that require acknowledgement from a physician that such text was reviewed. The management server 102 may further determine which medical tests or treatments are still need, as discussed above in connection with FIGS. 21 to 26. At this point, the clinical consultation note 160 is stored to the memory device 118 and ready for review and completion by a physician.

As shown in FIG. 27, the management server 102 transmits the clinical consultation note 160 to an application 152 on a clinician device 150 in response to a request message received from the device 150 (block 2714). The management server 102 next receives physician entries into the clinical consultation note (block 2716). Entries may be made pre-patient visit, during the patient visit, or after the patient visit. The entries include text typed by a physician into, for example, a physician exam section, a diagnosis/assessment section, a principal plan section, and/or an additional test/next actions section of the clinical consultation note 160, as discussed in connection with FIGS. 5 to 7. The management server 102 may also receive text that is copied from other sections.

The example management server 102 may receive a completion or submission indication from the clinician device 150. The management server 102 may analyze the clinical consultation note 160 to ensure all color-coded sections have been reviewed. If at least one section has not been reviewed, the management server 102 prevents submission of the note 160. In some instances, the management server 102 transmits a message indicative of which sections of the clinical consultation note 160 still needed to be reviewed or signed off. If the colored-coded sections have been reviewed (or there are no color-corded section), the management server 102 stores the clinical consultation note 160 in the memory device 118 (block 2718). This storage may also include writing the clinical consultation note 160 to an EMR of the patient.

As shown in FIG. 27, in some embodiments the management server 102 may cause physician-selected next actions to be performed (block 2720). After the actions are performed, the example procedure 2700 ends for the patient. In some embodiments, the management server 102 may use at least some of the information in the clinical consultation note 160 to prepopulate patient data 116 in the active sheet questionnaire 104 for another intake or admission for the same patient. Further, the medical documentation 140 stored in the memory device 118 is made available to the patient to select (or are automatically selected by the management server 102) for other clinical consultation notes 160.

The management server 102 may perform any one of the following actions. For example, the management server 102 may access a clinician calendar in a medical network and schedule a next appointment for a patient. The physician may specify a time frame or specific date for the next appointment in the clinical consultation note 160. The management server 102 may also transmit a message with medical prescription information to a pharmacy system. In this example, the physician may have provided a medication prescription in the Next Action section of the clinical consultation note 160. Further, the management server 102 may transmit a consult note to a referring physician. The management server may automatically generate a letter to the referring physician using at least some of the information from the clinical consultation note 160. In the Next Action section of the note 160, a physician may select a “copy primary care provider” option to cause the management server 102 to generate the letter.

The example management server 102 is also configured to transmit messages within a medical network to order diagnostic medical tests and/or medical treatments. The management server 102 may use a selected Next Action to place an automated EMR order entry, including one or more parameters for the testing or treatment. The management server 102 may use keyword-triggered instructions from the Next Action section and/or a planning section of the clinical consultation note 160 for translation in one or more EMR orders. The management server 102 may also convert the translation into a Health-Level 7 (“HL-7”) message. In an example, the management server 102 transcribes the text “CT abdomen and pelvis with IV contrast” from the Next Action section clinical consultation note 106 into a formal order for a CT image of the patient's abdomen and pelvis with an IV contrast.

The example management server 102 may also provide for automated billing based on completion of the clinical consultation note 160 and time spent conducting the consultation. The management server 102 may include a timer that records an amount of time a physician spent reviewing and entering text into a clinical consultation note 160. The management server 102 may also identify keywords from the note 160, which are translated into ICD-10 codes for billing. As an example, when the physician dictates “63 years old male with intermediate-risk prostate cancer” as their assessment in the clinical consultation note 160, management server 102 cross-links the medical information with the ICD-10 code C61, which is used in an EMR billing system. The time duration of the consultation or telehealth visit is used to bill the appropriate time-based telehealth code (99201-99205 for new or 99211-99215 for established, for example).

The management server 102 may further generate post-discharge home progress notes to a patient's EMR using the clinical consultation note 160. The management server 102 may select a relevant disease-specific post-discharge template that the patient will be prompted to fill out, enter salient photos, and plug biometric data at regular intervals post-operatively using the application 114 on the patient device 112. These templates are provided by the management server 102 to the application 114 on the patient device 112, and entered as automated “at-home progress notes” with pre-determined triggers for alerting the physician or representative (i.e., a high surgical drain output or a high pain score triggers an alert in an EMR system). The management server 102 may also provide patient home instructions as standardized written materials and patient education resources for diet, activity, and general do's and do not's, etc.

The management server 102 may additionally assign or provide for the viewing of written documents about standard national guidelines about their disease status for viewing in the application 114. The management server 102 may use specific active sheet responses (e.g “yes” to hematuria in urologic history) or triggering keywords from the clinical consultation note 160 to identify relevant patient education materials (e.g., links to videos and pamphlets). The application 114 may provide physician tracking to indicate whether the patient viewed the materials.

As provided above, the clinical consultation note 160 not only provides for reduced physician effort in documenting a patient's medical condition and next steps, the clinical consultation note 160 also provides a starting point for next steps in a patient's treatment. The automatic generation of scheduling, tests, and/or documentation reduces effort needed at the administration level to schedule and output such actions. Such a configuration of the management server 102 ensures that patient care does not accidently fall through systemic cracks.

### V. Medical Information Text/Image Extraction Embodiment

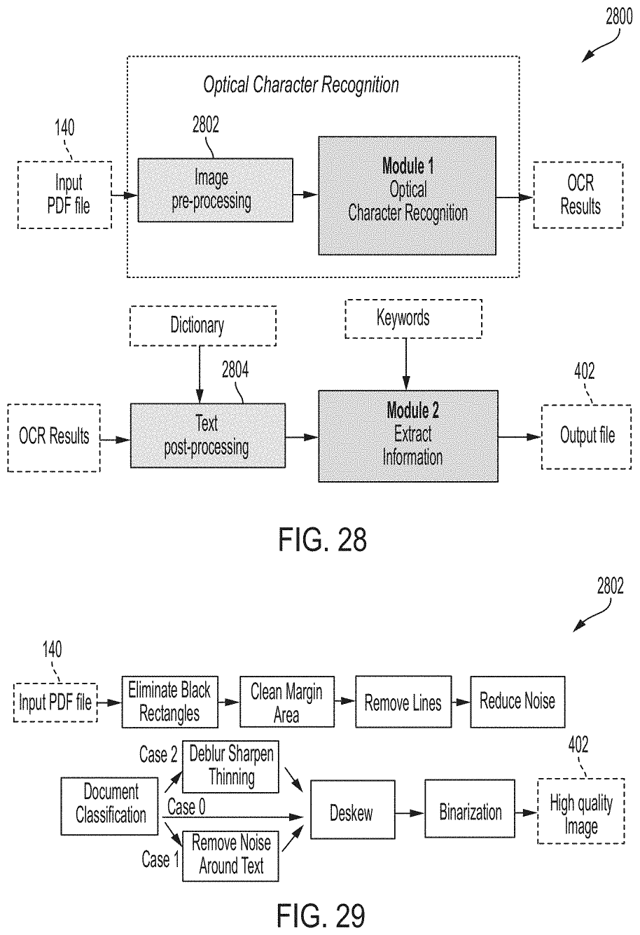

As discussed above, the medical information extractor 176 of the management server 102 is configured to extract relevant text and images (e.g., medical information 402) from medical documentation 140. FIG. 28 is a diagram of a data-in-taking process 2800, in accordance with an example embodiment of the present disclosure. Scanned or faxed medical documents 140 undergo an OCR process for conversion into searchable text documents. The process 2800 is configured to scan the medical documentation 140 using pre-determined processes and keywords to first identify and then extract the relevant aspects of laboratory, imaging, and/or pathology data into a database of the memory device 118 of FIG. 1. This pre-specified laboratory, imaging, and pathology data is customizable at a departmental and/or individual provider level.

The process 2800 includes an image pre-processing procedure 2802, which is show in more detail in FIG. 29. As shown in FIG. 29, the image pre-preprocessing procedure 2802 is configured to process images in an image file or an image embedded in a PDF file. The image pre-preprocessing procedure 2802 can include one or more algorithms/module to a) eliminate border and other bounding feature such as boxes, rectangle, etc.; b) clean margin area; c) remove lines; and/or d) and reduce image noise. The image pre-preprocessing procedure 2802 can also include one or more algorithms/modules to: a) classify a document; b) perform de-skewing; c) perform binarization; and/or d) sharpen and/or deblur image features and/or text. In some embodiments, the image pre-preprocessing procedure 2802 includes a de-skewing module (not shown) configured to detect a precise region of a single line, connect words in a line to a single area, rotate to almost horizontal (small angle remains), perform fine-tuning on connected area, and/or rotate to horizontal (i.e., eliminate remaining angle).

The management server 102 may also include a text processing module (not shown) configured to refine text at a word level and/or sentence level. For example, the text processing module can be configured to split connected words, replace “{”, “}”, “|”, “!” in words with “1”; and/or delete redundant spaces. Additionally, the text processing module can omit/correct numbers, expand acronyms, delete redundant characters, separate words and punctuations, fix special cases, remove lines by image, select real lines, and/or perform general document clean up.

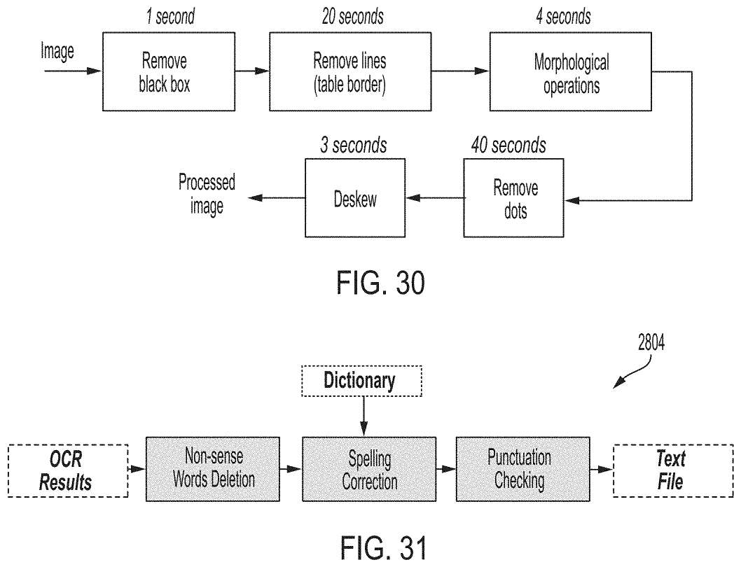

In some embodiments, the management server 102 is also configured to perform image processing, as shown in FIG. 30. Additionally, as shown, each step can be done in the time indicated or faster, depending on the available hardware resources. The image processing can include removing black boxes, removing lines or table borders, performing morphological operations, removing dots, and/or de-skewing an image.

After OCR is completed, a quality checking process 2804 can be performed. FIG. 31 illustrates a diagram of a quality checking process 2804 that can include one or more functions to delete non-sense words, correct spellings, and/or correct punctuations and other grammatical errors. Once these steps are completed, the process 2804 can output a text file of the results. In some embodiments, the process 2804 is configured to perform post-processing word correction, as shown in FIG. 28, by using dictionary/glossary for units and exemption cases for splitting before text-extraction using keyword matching. The digit correction process of the process 2804 can use various architectures such as, but not limited to, Tesseract, LSTM, neural network, and CNN, which may be configured to focus on medical digits with an enhanced model that extends to all numbers. When OCR is unsure about the accuracy of a particular extracted letter or number, it will color that specific letter or number in a different color, say red, in order to alert the physician and draw the physician's attention to check the source document for accuracy.

The example process 2804 may also be configured to perform data distillation using cut patches, boundary extension, and/or feature extraction decision. The process 2804 is configured to generate an enhanced model using data washing, boundary extension, feature extraction decision, and/or removing hard case noise by de-noising based on symmetric checking (e.g., Heuristic de-noising). The process 2804 may also be configured to generate visualized confidence by using a color-coded depiction of correct versus incorrect words in the medical documentation 140 based on high versus low confidence of an OCR read-out.

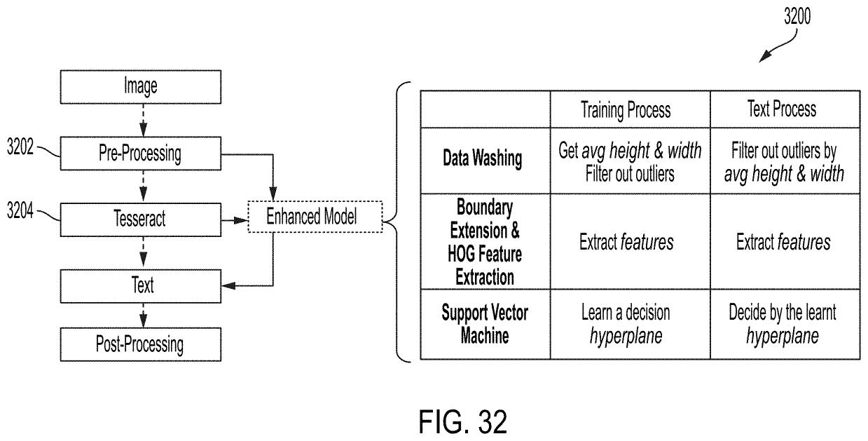

FIG. 32 is a diagram of an image processing procedure/process 3200, according to an example embodiment of the present disclosure. The process 3200 includes a pre-processing sub-process 3202 that generate an enhance model using one or more tools such as, but not limited to, data washing, histogram of oriented gradients (“HOG”) feature extraction, and/or support vector machine. The data washing module can be configured to filter out outliers using at least average height and width. Sophisticated modifications of a Tesseract engine 3204 can also be used to generate the enhanced model. Tesseract is an open source AI-based OCR platform. It should be noted that other OCR platforms or algorithms can also be used. Finally, outputs from the enhanced module undergo post-processing correction(s) based on dictionaries/glossaries to output clean and enhanced text data.

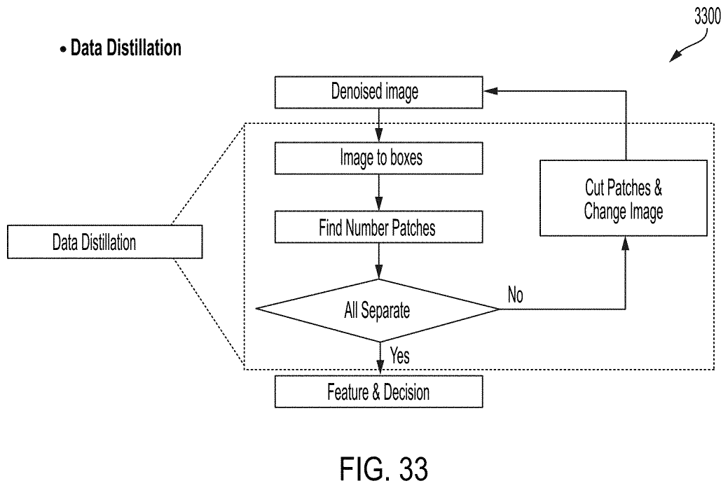

FIG. 33 is a diagram of an example data distillation process 3300, according to an example embodiment of the present disclosure. The process 3300 is configured to at least de-noise an image, find number patches, cut and separate patches, and/or locate relevant features, such as pre-determined keywords, larger font size words, and/or professional logos of outside labs/facilities, etc.

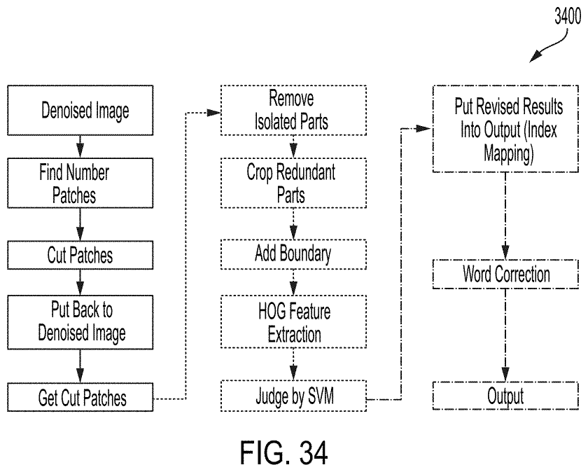

FIG. 34 is a diagram of a process 3400 for processing image and/or text, according to an example embodiment of the present disclosure. As shown, the process 3400 includes one or more sub-processes to de-noise an image, locate number patches, cut patches (thereby increasing the accuracy in identifying numerical images and numbers), place patches back to de-noise an image, get cut patches, remove isolated parts, crop redundant parts, add a boundary, extract a histogram of oriented gradients (“HOG”) feature, judge by support vector machines (“SVM”), perform index mapping, perform word correcting, and/or output a text file. It should be noted that one or more sub-processes (e.g., boxes, steps) shown in FIG. 34 can be omitted, skipped, or rearranged in any order. The histogram of oriented gradients (“HOG”) feature descriptor is used to extract features from image data by focusing on a structure/shape of text to extract edge gradient/orientation for providing edge identification/direction. This is calculated in localized portions by breaking the image into smaller regions and calculating gradients/orientation and histogram for each region separately.

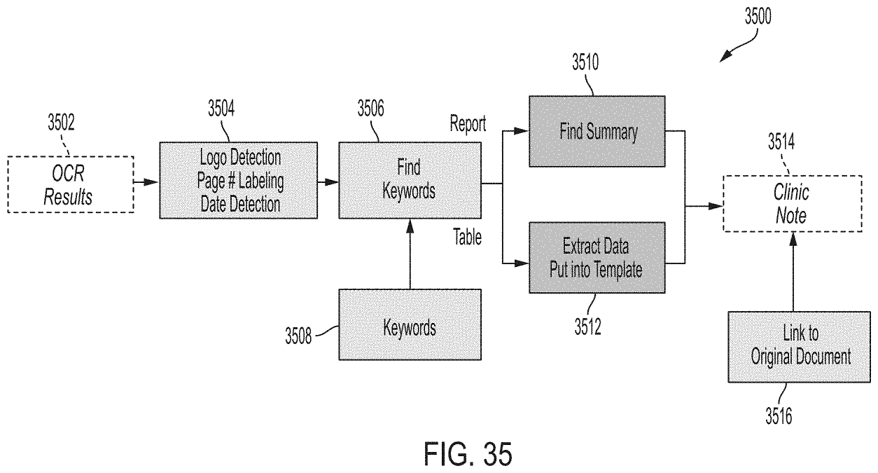

FIG. 35 is a diagram of a process 3500 to generate a clinical consultation note 160, according to an example embodiment of the present disclosure. The process 3500 beings by taking an OCR result 3502 and performing at least logo detection, page number labeling, and date detection (block 3504). Next, the example process 3500 performs keyword searching using a list of keywords 3508 (block 3506). At sub-processes 3510 and 3512, data is extracted, and a summary is generated. Next, at block 3514, the clinical consultation note 160 is generated based on output(s) from one or more sub-processes 3404, 3506, 3510, and 3512. The process 3500 may also create a link in the clinical consultation note 160 to original medical documentation 140 (block 3516).

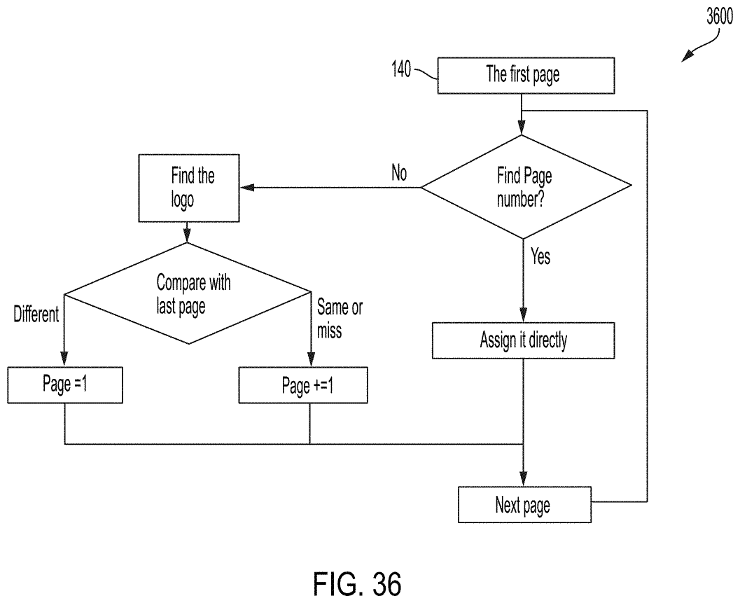

FIG. 36 is a diagram of an exemplary page number labeling OCR process 3600, according to an example embodiment of the present disclosure. As shown, the process 3600 can use logo detection to identify pages. This could help with data organization and ensuring that data is not presented in a wrong order. This data organization and page order specifically pertains to OCR-extracted data from scanned-in records of outside lab/imaging reports of the medical documentation 140. Presenting this data in the correct temporal order is important and may be accomplished in two ways. For example, various lab/radiology reports are presented in the clinical consultation note 160 sequentially in a chronologic order, according to the date performed, i.e., tests performed on an earlier date first, followed by subsequent dates. In another example, for faxed medical documentation 140 that is longer than 1 page (e.g., 2-3 pages), it is important that these pages be presented in the correct sequence. For instance, for a 2-page lab report, the OCR process 3600 searches for the keywords “1 of 2 pages” and “2 of 2 pages”, thereby correctly identifying their ordered sequence. This is important for correctly extracting data from various test results.

In an example for a CT scan report, the process 2800 is configured to extract a Summary/Conclusion data-points from the CT scan report and insert this into the pre-allotted sections or slots of the clinical consultation note 160. To achieve this, the OCR process 3600 first locates the page(s) titled “CT scan report”, and then identifies if it is a 1- or a 2-page report. The OCR process 3600 then identifies the appropriate “Summary” or “Conclusion” section, which is typically located on the last page of that CT scan report. The OCR process 3600 then extracts this summary/conclusion information and inserts it into the pre-formatted section for a CT scan in the clinical consultation note 160.

### VI. Billing Embodiment

As discussed above, the management server 102 is configured, in some embodiments, to provide for automated billing. In an example, the clinical consultation note 160 may provide the following statements (a) to (p):

_Management of an uncomplicated self-limiting illness or injury (a)
_Management of 2 or more self-limiting illnesses or minor problems (b)
_Management of an acute illness with systemic symptoms (c)
_Management of an acute complicated injury (d)
_Management of one stable chronic illness only (e)
_Management of one or more stable chronic illnesses (f)
_Management of one or more chronic illnesses with exacerbation, progression, or treatment side effects (g)
_Management of one or more chronic illnesses with severe exacerbation, progression, or treatment side effects (h)
_Management of new problem with uncertain prognosis (i)
_Management of two stable chronic illnesses (j)
_Review of prior external note(s) (k)
_Review of laboratory or imaging results (1)
_Ordering of diagnostic tests (m)
_Obtained history from a surrogate (n)
_Independently interpreting tests performed by another physician or health care provider (o)
_Discussion of management or test interpretation with external physician (p)

The clinical consultation note 160 receives a selection of some of these statements from the physician based on the nature of the consultation. Selection of these statements is used by the management server 102 to recommend a billing level (e.g., 99201/11-99205-15) based on medical decision making versus a time requirement (captured by internal timer in the application 152 and/or the management server 102). The internal timer may record an amount of time a physician spent reviewing and/or completing the clinical consultation note 160.

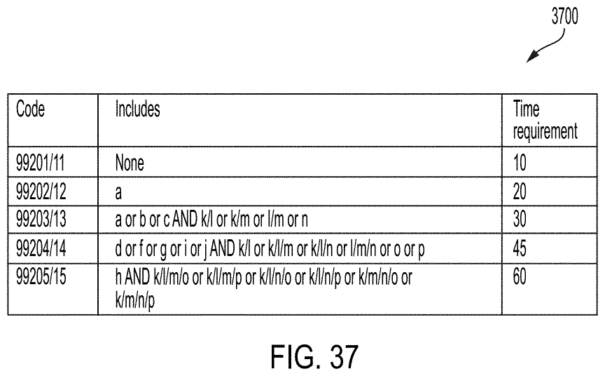

FIG. 37 is a diagram of a chart 3700 that shows a time requirement for certain billing labels and the corresponding statements (a) to (p), according to an example embodiment of the present disclosure. The example management server 102 is configured to determine which billing code is higher (either time-based or medical decision-making). The management server 102 then recommends or outputs the higher of the billing codes for instance.

### VII. Conclusion

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A system for automatically pre-constructing a clinical consultation note during a patient intake/admission process, the system comprising:
a memory device storing an active sheet including a medical questionnaire form having data fields that prompt a patient to select symptoms from among a plurality of symptoms, the active sheet including embedded logic configured to:
select detailed medical questions based on the selection of symptoms, the detailed medical questions corresponding to one or more possible disease conditions, and
determine tests or medical documentation needed for a consultation with a physician; and
a server communicatively coupled to the memory device, the server configured to:
receive a request from a patient device to schedule a medical appointment with a physician,
transmit the active sheet to the patient device after receiving the request,
receive patient data from the patient device via the active sheet, the patient data including a selection of symptoms and answers to the detailed medical questions,
receive medical documentation via the active sheet,
extract relevant medical information from the medical documentation, the medical information including at least one of test results, medical images, clinician notes, prior treatments, or medical history information,
generate automatically a clinical consultation note using the extracted relevant medical information and at least some of the patient data from the active sheet, and
transmit the clinical consultation note to a physician device for the medical appointment with the patient.

2. The system of claim 1, wherein the server is further configured to:
receive, via the active sheet, information indicative of another medical provider;
transmit a request for third-party medical documentation to the other medical provider;
receive the third-party medical documentation from the other medical provider; and
extract relevant medical information from the third-party medical documentation for inclusion within the clinical consultation note.

3. The system of claim 1, wherein the active sheet is configured to prompt a patient for additional patient data including at least one of past medical history, past surgical history, current/past medications, current/past allergies, family and social history, history of present illness, or general activity level.

4. The system of claim 1, wherein the server is further configured to select the detailed medical questions embedded within the active sheet also using the additional medical information.

5. The system of claim 1, wherein the test or medical documentation includes at least one of x-rays, patient fluid laboratory results, blood test laboratory results, computed tomography (“CT”) images and/or reports, magnetic resonance imaging (“MRI”) images and/or reports, ultrasound images and/or reports, previous clinical notes, or an electronic medical record (“EMR”).

6. The system of claim 1, wherein the patient device includes an application configured as the active sheet, the application configured to receive the patient data from the patient and transmit the patient data to the server via one or more application programming interfaces.

7. The system of claim 1, wherein the clinical consultation note includes sections for a review of patient symptoms, identification of a referring physician, identification of a pharmacy, laboratory results, medical images, prior clinical notes, a diagnosis/assessment to be completed by the physician, a principal plan to the completed by the physician, additional tests to be completed or selected by the physician, a discussion of risks benefits to be completed by the physician, an impression of medical condition, or action items to be completed.

8. The system of claim 7, wherein the server is configured to:
receive the clinical consultation note from the physician device with sections completed by the physician and at least one action item selection; and
store the clinical consultation note to a medical record associated with the patient.

9. The system of claim 7, wherein the server is further configured to cause a medical system to perform the at least one action item selected.

10. The system of claim 7, wherein the at least one action item includes at least one of a medication order, a request to send the clinical consultation note to another physician, an order for a laboratory test, a schedule for a next appointment, a request to send standardized written materials related to a medical condition to the patient device, or a request to submit a bill.

11. The system of claim 7, wherein the server is further configured to:
cause at least some of the sections of the clinical consultation note to be colored coded; and
remove or change the color coding after detecting or receiving an indication that the section was selected or reviewed by the physician.

12. The system of claim 1, wherein the server is further configured to generate automatically the clinical consultation note by embedding at least some links in the clinical consultation note to the related extracted relevant medical information, wherein selection of an embedded link causes the related extracted relevant medical information to be displayed in conjunction with the clinical consultation note.

13. The system of claim 1, wherein the server is further configured to generate automatically the clinical consultation note by embedding at least some links in the clinical consultation note to the at least some of the patient data from the active sheet.

14. The system of claim 1, wherein the server is further configured to extract the relevant medical information from the medical documentation by performing one or more of the following functions: denoise the medical documentation, find number patches in the medical documentation, cut patches from the medical documentation, identify a denoised image in the medical documentation, remove isolated parts of the medical documentation, crop redundant or unneeded parts of the medical documentation, add a boundary to the medical documentation, or extract features from the medical documentation.

15. The system of claim 1, wherein the server is further configured to extract the relevant medical information from the medical documentation by performing one or more of the following functions: remove black boxes, remove a table border, remove dots, or de-skew at least portions of the medical documentation.

16. The system of claim 1, wherein the server is further configured to extract the relevant medical information from the medical documentation by modifying words and punctuation by deleting non-sense words, correcting spelling, or correcting punctuation.

17. The system of claim 1, wherein at least some of the patient data is provided by a peripheral device that is communicatively coupled to the patient device, the peripheral device including at least one of a fitness tracker, a smartwatch, a smart-ring, a wireless weight scale, or a blood pressure cuff.

18. The system of claim 1, wherein the server is further configured to:
use a timer system to determine an aggregate time spent a) pre-visit filling out the active sheet on behalf of the patient, b) intra-visit during the actual medical appointment with the patient, and c) post-visit spent completing the clinical consultation note; and
cause a bill to be generated based on the aggregate time.

19. The system of claim 1, wherein the server is further configured to:
receive a second request from the patient device to schedule a subsequent medical appointment with the physician; and
use at least some of the patient data to pre-construct the active sheet.

20. The system of claim 1, wherein the server is further configured to receive additional medical documentation from a remote electronic medical record system via interoperability.

21. A method for automatically pre-constructing a clinical consultation note during a patient intake/admission process, the method comprising:
receiving, in a server, a request from a patient device to schedule a medical appointment with a physician;
transmitting, from the server to the patient device, an active sheet after receiving the request, the active sheet being stored in a memory device, the active sheet including an electronic medical questionnaire form having data fields that prompt a patient to select symptoms from among a plurality of symptoms, the active sheet including embedded logic configured to:
select detailed medical questions based on the selection of symptoms, the detailed medical questions corresponding to one or more possible disease conditions, and
determine tests or medical documentation needed for a consultation with a physician;
receiving, in the server, patient data from the patient device via the active sheet, the patient data including a selection of symptoms and answers to the detailed medical questions;
receiving, in the server, medical documentation via the active sheet;
extracting, via the server, relevant medical information from the medical documentation, the medical information including at least one of test results, medical images, clinician notes, prior treatments, or medical history information;
generating automatically, via the server, a clinical consultation note using the extracted relevant medical information and at least some of the patient data from the active sheet; and
transmitting, via the server, the clinical consultation note to a physician device for the medical appointment with the patient.

22. The method of claim 21, further comprising:
receiving, in the server via the active sheet, information indicative of another medical provider;
transmitting, from the server, a request for third-party medical documentation to the other medical provider;
receiving, in the server, the third-party medical documentation from the other medical provider; and
extracting, via the server, relevant medical information from the third-party medical documentation for inclusion within the clinical consultation note.

23. The method of claim 21, further comprising:
receiving, in the server, the clinical consultation note from the physician device with sections completed by the physician and at least one action item selection;
storing, via the server, the clinical consultation note to a medical record associated with the patient; and
causing, via the server, a medical system to perform the at least one action item selected,
wherein the at least one action item includes at least one of a medication order, a request to send the clinical consultation note to another physician, an order for a laboratory test, a schedule for a next appointment, a request to send standardized written materials related to a medical condition to the patient device, or a request to submit a bill.
